SEQUENCE LISTING (114 sequences)

SEQ ID NO: 1 - protein, 506 aa, synthetic construct
  UNSURE 1..4: note = optionally absent
ATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGV

SEQ ID NO: 2 - protein, 155 aa, synthetic construct
  UNSURE 1..2: note = optionally absent
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 3 - protein, 155 aa, synthetic construct
  UNSURE 1..2: note = optionally absent
MGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 4 - protein, 152 aa, synthetic construct
  UNSURE 1..2: note = optionally absent
MGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 5 - protein, 565 aa, synthetic construct
  UNSURE 1: note = optionally absent
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 6 - protein, 7 aa, synthetic construct
IEHIENE

SEQ ID NO: 7 - protein, 18 aa, synthetic construct
IEDKIEEILSKIYHIENE

SEQ ID NO: 8 - protein, 19 aa, synthetic construct
NIENKIEEIEKKIYHIENE

SEQ ID NO: 9 - protein, 19 aa, synthetic construct
EIEKKIEEIEKKIERIENE

SEQ ID NO: 10 - protein, 19 aa, synthetic construct
NINKKIEEIEKKIYHIENE

SEQ ID NO: 11 - protein, 18 aa, synthetic construct
INKKIEEIEKKIYHIENE

SEQ ID NO: 12 - protein, 20 aa, synthetic construct
EEINKKIEEIEKKIYHIENE

SEQ ID NO: 13 - protein, 14 aa, synthetic construct
IENINSKIYHIENE

SEQ ID NO: 14 - protein, 14 aa, synthetic construct
IEAILSKIYHIENE

SEQ ID NO: 15 - protein, 14 aa, synthetic construct
IENIASKIYHIENE

SEQ ID NO: 16 - protein, 14 aa, synthetic construct
IEQILRKIYHIENE

SEQ ID NO: 17 - protein, 25 aa, synthetic construct
IEDKIEEILRKIEEIEKKIYHIENE

SEQ ID NO: 18 - protein, 27 aa, synthetic construct
EEIKDKIEEILRKIEEIEKKIYHIENE

SEQ ID NO: 19 - protein, 18 aa, synthetic construct
ILSKIYHIERKIEEILNE

SEQ ID NO: 20 - protein, 18 aa, synthetic construct
IEDKIYHIERKIEEILNE

SEQ ID NO: 21 - protein, 19 aa, synthetic construct
NIEDKIYHIERKIEEILNE

SEQ ID NO: 22 - protein, 17 aa, synthetic construct
LSKIYHIERKIEEILNE

SEQ ID NO: 23 - protein, 39 aa, synthetic construct
ILSKIYHIEDKIEEINRKIEHILSKIYHIERKIEEILNE

SEQ ID NO: 24 - protein, 39 aa, synthetic construct
IEDKIYHIEDKIEEINRKIEHILSKIYHIERKIEEILNE

SEQ ID NO: 25 - protein, 40 aa, synthetic construct
NIEDKIYHIEDKIEEINRKIEHILSKIYHIERKIEEILNE

SEQ ID NO: 26 - protein, 42 aa, synthetic construct
IENINSKIYHIEDKIEEINRKIEHILSKIYHIERKIEEILNE

SEQ ID NO: 27 - protein, 46 aa, synthetic construct
IEDKIEEILSKIYHIEDKIEEINRKIEHILSKIYHIERKIEEILNE

SEQ ID NO: 28 - protein, 214 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGF

SEQ ID NO: 29 - protein, 213 aa, synthetic construct
IAPLQLGKCNIAGWLLGNPECDLLLTASSWSYIVETSNSENGTCYPGDFIDYEELRCQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNR

SEQ ID NO: 30 - protein, 214 aa, synthetic construct
IAPLQLGKCNIAGWLLGNPECDPLLSVRSWSYIVETPNSENGICYPGDFIDYEELRCQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALRRGF

SEQ ID NO: 31 - protein, 214 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGF

SEQ ID NO: 32 - protein, 215 aa, synthetic construct
VAPLHLGKCNIAGWILGNPECESLSTASSWSYIVETSNSDNGTCFPGDFINYEELRCQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNA

SEQ ID NO: 33 - protein, 213 aa, synthetic construct
IAPLQLGNCSIAGWLLGNPECDLLLNASSWSYIVETSNSENGTCYPGDFIDYEELRCQLSSVSSFEKFEIFPKNSSWPNHTTTKGVTAACSYAGASSFYRNLLWLTKKGSSYPKLSKNYTNNKGKEVLVLWGVHHPPTGTDQQSLYQNADAYVSVGSSKYNRRFTPEIAARPKVRDQAGRMNYYWTLLEPGDTITFEATGNLIAPWYAFALNR

SEQ ID NO: 34 - protein, 214 aa, synthetic construct
IAPLQLGNCSIAGWLLGNPECDPLLNVSSWSYIVETPNSENGTCYPGDFIDYEELRCQLSSVSSFERFEIFPKNSSWPNHTTNGVTAACSHEGKSSFYRNLLWLTEKEGSYPNLTNNYTNKKGKEVLVLWGIHHPPNSKEQQNLYQNENAYVSVVTSNYNRRFTPEIAERPKVRDQAGRMNYYWTLLKPGDTIIFEANGNLIAPMYAFALRRGF

SEQ ID NO: 35 - protein, 214 aa, synthetic construct
IAPLQLGNCSVAGWILGNPECNLSINASSWSYIVETPNPENGTCFPGYFADYEELRCQLSSVSSFERFEIFPKNSSWPNHTVTGVSASCSHNGKSSFYRNLLWLTGKNGLYPNLSKNYTNNKSKEVLVLWGVHHPPNIGNQRALYHTENAYVSVVSSHYSRRFTPEIAKRPKVRDQEGRINYYWTLLEPGDTIIFEANGNLIAPWYAFALSRGF

SEQ ID NO: 36 - protein, 215 aa, synthetic construct
VAPLHLGNCSIAGWILGNPECESLNTSSNWSYIVETSNSDNGTCFPGDFINYEELRCQLSSVSSFERFEIFPKNSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNA

SEQ ID NO: 37 - protein, 213 aa, synthetic construct
IAPLQLGKCNIAGWLLGNPECDLLLTASSWSYIVETSNSENGTCYPGDFIDYEELRCQLSSVSSFEKFEIFPKNSSWPNHETTKGVTAACSYAGASSFYRNLLWLTKKGSSYPKLSKSYVNNKGKEVLVLWGVHHPPTGTDQQSLYQNEDAYVIVASSKYDRVFTPLIAARPKVRDQAGRMNYYWTLLEPGDTITFEATGNLIAPWYAFALNR

SEQ ID NO: 38 - protein, 214 aa, synthetic construct
IAPLQLGNCSIAGWLLGNPECDPLLSVRSWSYIEETPNSENGICYPGDFIDYEELRCQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVFVGTSNYDRIFTPIIAERPKVRDQAGRMNYYWTLLKPGDTIIFEANGNLIAPMYAFALRRGF

SEQ ID NO: 39 - protein, 214 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRVFTPIIAKRPKVRDQEGRINYYWTLLEPGDTIIFEANGNLIAPWYAFALSRGF

SEQ ID NO: 40 - protein, 214 aa, synthetic construct; identical to SEQ ID NO: 39

SEQ ID NO: 41 - protein, 214 aa, synthetic construct; identical to SEQ ID NO: 39

SEQ ID NO: 42 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYDRVFTPIIVKRPKVRDQEGRINYYWTLLEPGDTIIFEANGNLIAPWYAFALSRGFG

SEQ ID NO: 43 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYSREFTPVIVKRPKVRDQEGRINYYWTLLEPGDTIIFEANGNLIAPWYAFALSRGFG

SEQ ID NO: 44 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYDRRFTPQIVKRPKVRDQEGRINYYWTLLEPGDTIIFEANGNLIAPWYAFALSRGFG

SEQ ID NO: 45 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRRFTPQIAKRPKVRDQEGRINYYWTLLEPGDTIIFEANGNLIAPWYAFALSRGFG

SEQ ID NO: 46 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVAVVSSHYSRVFTPIILKRPKVRDQEGRINYYWTLLEPGDTIIFEANGNLIAPWYAFALSRGFG

SEQ ID NO: 47 - protein, 215 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVAVVSSHYDRVFTPTILKRPKVRDQEGRINYYWTLLEPGDTIIFEANGNLIAPWYAFALSRGFG

SEQ ID NO: 48 - protein, 215 aa, synthetic construct
VAPLHLGKCNIAGWILGNPECESLSTASSWSYIVETSNSDNGTCFPGNFINYEELRCQLSSVSSFERFEIFPKTSSWPNHDSNKGVTAACPHAGAKSFYKNLIWLVKKGNSYPKLNQSYINDKGKEVLVLWGIHHPSTTADQQSLYQNEDTYVFVSTSRYDKVFKPIIATRPKVRDQEGRMNYYWTLVEPGDKITFEATGNLVVPRYAFTMERNA

SEQ ID NO: 49 - protein, 213 aa, synthetic construct
IAPLQLGNCSIAGWLLGNPECDLLLNASSWSYIVETSNSENGTCYPGDFIDYEELRCQLSSVSSFEKFEIFPKNSSWPNHTTTKGVTAACSYAGASSFYRNLLWLTKKGSSYPKLSKNYTNNKGKEVLVLWGVHHPPTGTDQQSLYQNEDAYVIVASSKYDRVFTPLIAARPKVRDQAGRMNYYWTLLEPGDTITFEATGNLIAPWYAFALNR

SEQ ID NO: 50 - protein, 214 aa, synthetic construct
IAPLQLGNCSIAGWLLGNPECDPLLNVSSWSYIEETPNSENGTCYPGDFIDYEELRCQLSSVSSFERFEIFPKNSSWPNHTTNGVTAACSHEGKSSFYRNLLWLTEKEGSYPNLTNNYTNKKGKEVLVLWGIHHPPNSKEQQNLYQNENAYVFVGTSNYDRIFTPIIAERPKVRDQAGRMNYYWTLLKPGDTIIFEANGNLIAPMYAFALRRGF

SEQ ID NO: 51 - protein, 212 aa, synthetic construct
IAPLQLGNCSVAGWILGNPECNLSINASSWSYIVETPNPENGTCFPGYFADYEELRCQLSSVSSFERFEIFPKNSSWPNHTVTGVSASCSHNGKSSFYRNLLWLTGKNGLYPNLSKNYTNNKSKEVLVLWGVHHPPNIGNQRALYHTENAYVLVVSSHYDRVFTPIIAKRPKVRDQEGRINYYWTLLEPGDTIIFEANGNLIAPWYAFALSR

SEQ ID NO: 52 - protein, 215 aa, synthetic construct
VAPLHLGNCSIAGWILGNPECESLNTSSNWSYIVETSNSDNGTCFPGNFINYEELRCQLSSVSSFERFEIFPKNSSWPNHDSNKGVTAACPHAGAKSFYKNLIWLVKKGNSYPKLNQSYINDKGKEVLVLWGIHHPSTTADQQSLYQNEDTYVFVSTSRYDKVFKPIIATRPKVRDQEGRMNYYWTLVEPGDKITFEATGNLVVPRYAFTMERNA

SEQ ID NO: 53 - protein, 237 aa, synthetic construct
EKRGKLCPKCLNCTDLDVALGRPKCTGKIPSARTSELHEERPVTSGCFPIRHDLTKIRCLPNLLRGYEHIRLSTHNVINAENAPGGPYKIGTSGSCPNITNGNGFFATMAWAVPKNDKNKTATEPLTIEVPYICTEGEDQITVWGFHSDNETQMAKLYGDSKPQQFTSIANGVTTHYVSQIGGFPNQTEDGGLPQSGRIVVDYMVQKSGKTGIIIYQRGILLPQKVWCASGRSKVIK

SEQ ID NO: 54 - protein, 236 aa, synthetic construct
RTRGKLCPDCLNCTDLDVALGRPECVGTTPSAKTSELHEREPVTSGCFPIRHDLTKIRCLPNLLRGYEKIRLSTQNVIDAEKAPGGPYRLGTSGSCPNATSKIGFFATMAWAVPKDNYKNATEPLTVEVPYICTEGEDQITVWGFHSDNKTQMKSLYGDSKPQQFTSIANGVTTHYVSQIGDFPDQTEDGGLPQSGRIVVDYMMQKPGKTGIIIYQRGVLLPQKVWCASGRSKVTK

SEQ ID NO: 55 - protein, 206 aa, synthetic construct
KQLDGEDCTLIDALLGDPHCDGFQNETWDQFTERSKAYSNCYPYDVPDYESLRCLVAESGTLEFITEGFTWTGVTQNGGSNACKRNGESGFFSRLNWLTKSGSTYPVLNVTMPNNDNFDKLYIWGVHHPSTNQEQKSLYVQESGRVTVSTRRSQQTINPNIGSRPWVRGLSSRISIYYTIVKPGDVLTINSNGNLIAPRGYFKMKT

SEQ ID NO: 56 - protein, 215 aa, synthetic construct
VKPLQLRDCSVAGWLLGNPECDEFINVSEWSYIVEKENPTNDLCFPGSFNDYEELKCLLSAINHFEKIQIIPKSSWSDHEASSGVSSACPYLGSPSFFRNVVWLIKKNSTYPTIKKSYNNTNQEDLLVLWGIHHPNDAAEQTRLYQNPTTYIIIGTSTLDQVLTPVIATRPKVNGQSGRMEFFWTILKPNDAIIFESNGNFIAPEYAYKIVKKGD

SEQ ID NO: 57 - protein, 212 aa, synthetic construct
IKPLELGDCSIAGWLLGNPECDRLLSVSEWSYITEKENPRDGLCYPGSFNDYEELKCLLSSVKHFEKVKILPKDRWTQHTTTGGSRACAVSGNPSFFRNMVWLTEKGSNYPVAKGSYNNTSGEQMLIIWGVHHPNDETEQRTLYQNVGTYVVVGTSTLDKVSTPIIATRPKVNGQGGRMEFSWTLLDMWDTIIFESTGNLIAPEYGFKISKR

SEQ ID NO: 58 - protein, 125 aa, synthetic construct
IEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 59 - protein, 136 aa, synthetic construct
IEDKIEEILSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 60 - protein, 137 aa, synthetic construct
NIENKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 61 - protein, 137 aa, synthetic construct
EIEKKIEEIEKKIERIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 62 - protein, 137 aa, synthetic construct
NINKKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 63 - protein, 136 aa, synthetic construct
INKKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 64 - protein, 138 aa, synthetic construct
EEINKKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 65 - protein, 132 aa, synthetic construct
IENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 66 - protein, 132 aa, synthetic construct
IEAILSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 67 - protein, 132 aa, synthetic construct
IENIASKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 68 - protein, 132 aa, synthetic construct
IEQILRKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 69 - protein, 143 aa, synthetic construct
IEDKIEEILRKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 70 - protein, 145 aa, synthetic construct
EEIKDKIEEILRKIEEIEKKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 71 - protein, 136 aa, synthetic construct
ILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 72 - protein, 136 aa, synthetic construct
IEDKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 73 - protein, 137 aa, synthetic construct
NIEDKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 74 - protein, 135 aa, synthetic construct
LSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 75 - protein, 157 aa, synthetic construct
ILSKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 76 - protein, 157 aa, synthetic construct
IEDKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 77 - protein, 158 aa, synthetic construct
NIEDKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 78 - protein, 160 aa, synthetic construct
IENINSKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 79 - protein, 164 aa, synthetic construct
IEDKIEEILSKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 80 - protein, 8 aa, synthetic construct
GSGSGSGS

SEQ ID NO: 81 - protein, 5 aa, synthetic construct
GSGSG

SEQ ID NO: 82 - protein, 12 aa, synthetic construct
GSGSGSGECHSP

SEQ ID NO: 83 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGSGKRIENILSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 84 - protein, 347 aa, synthetic construct
IAPLQLGKCNIAGWLLGNPECDLLLTASSWSYIVETSNSENGTCYPGDFIDYEELRCQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 85 - protein, 346 aa, synthetic construct
IAPLQLGKCNIAGWLLGNPECDPLLSVRSWSYIVETPNSENGICYPGDFIDYEELRCQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALRRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 86 - protein, 346 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 87 - protein, 347 aa, synthetic construct
VAPLHLGKCNIAGWILGNPECESLSTASSWSYIVETSNSDNGTCFPGDFINYEELRCQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 88 - protein, 347 aa, synthetic construct
IAPLQLGNCSIAGWLLGNPECDLLLNASSWSYIVETSNSENGTCYPGDFIDYEELRCQLSSVSSFEKFEIFPKNSSWPNHTTTKGVTAACSYAGASSFYRNLLWLTKKGSSYPKLSKNYTNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 89 - protein, 346 aa, synthetic construct
IAPLQLGNCSIAGWLLGNPECDPLLNVSSWSYIVETPNSENGTCYPGDFIDYEELRCQLSSVSSFERFEIFPKNSSWPNHTTNGVTAACSHEGKSSFYRNLLWLTEKEGSYPNLTNNYTNKKGKEVLVLWGIHHPPNSKEQQNLYQNENAYVSVVTSNYNRRFTPEIAERPKVRDQAGRMNYYWTLLKPGDTIIFEANGNLIAPMYAFALRRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 90 - protein, 346 aa, synthetic construct
IAPLQLGNCSVAGWILGNPECNLSINASSWSYIVETPNPENGTCFPGYFADYEELRCQLSSVSSFERFEIFPKNSSWPNHTVTGVSASCSHNGKSSFYRNLLWLTGKNGLYPNLSKNYTNNKSKEVLVLWGVHHPPNIGNQRALYHTENAYVSVVSSHYSRRFTPEIAKRPKVRDQEGRINYYWTLLEPGDTIIFEANGNLIAPWYAFALSRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 91 - protein, 347 aa, synthetic construct
VAPLHLGNCSIAGWILGNPECESLNTSSNWSYIVETSNSDNGTCFPGDFINYEELRCQLSSVSSFERFEIFPKNSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 92 - protein, 347 aa, synthetic construct
IAPLQLGKCNIAGWLLGNPECDLLLTASSWSYIVETSNSENGTCYPGDFIDYEELRCQLSSVSSFEKFEIFPKNSSWPNHETTKGVTAACSYAGASSFYRNLLWLTKKGSSYPKLSKSYVNNKGKEVLVLWGVHHPPTGTDQQSLYQNEDAYVIVASSKYDRVFTPLIAARPKVRDQAGRMNYYWTLLEPGDTITFEATGNLIAPWYAFALNRGSGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 93 - protein, 346 aa, synthetic construct
IAPLQLGNCSIAGWLLGNPECDPLLSVRSWSYIEETPNSENGICYPGDFIDYEELRCQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVFVGTSNYDRIFTPIIAERPKVRDQAGRMNYYWTLLKPGDTIIFEANGNLIAPMYAFALRRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 94 - protein, 346 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRVFTPIIAKRPKVRDQEGRINYYWTLLEPGDTIIFEANGNLIAPWYAFALSRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 95 - protein, 353 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRVFTPIIAKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 96 - protein, 381 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRVFTPIIAKRPKVRDQEGRINYYWTLLEPGDTIIFEANGNLIAPWYAFALSRGFGSGSGSCIENINSKIYHIEDKIEEINRKIEHILSKIYHIERKIEEILNEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 97 - protein, 347 aa, synthetic construct
VAPLHLGKCNIAGWILGNPECESLSTASSWSYIVETSNSDNGTCFPGNFINYEELRCQLSSVSSFERFEIFPKTSSWPNHDSNKGVTAACPHAGAKSFYKNLIWLVKKGNSYPKLNQSYINDKGKEVLVLWGIHHPSTTADQQSLYQNEDTYVFVSTSRYDKVFKPIIATRPKVRDQEGRMNYYWTLVEPGDKITFEATGNLVVPRYAFTMERNAGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 98 - protein, 347 aa, synthetic construct
IAPLQLGNCSIAGWLLGNPECDLLLNASSWSYIVETSNSENGTCYPGDFIDYEELRCQLSSVSSFEKFEIFPKNSSWPNHTTTKGVTAACSYAGASSFYRNLLWLTKKGSSYPKLSKNYTNNKGKEVLVLWGVHHPPTGTDQQSLYQNEDAYVIVASSKYDRVFTPLIAARPKVRDQAGRMNYYWTLLEPGDTITFEATGNLIAPWYAFALNRGSGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 99 - protein, 346 aa, synthetic construct
IAPLQLGNCSIAGWLLGNPECDPLLNVSSWSYIEETPNSENGTCYPGDFIDYEELRCQLSSVSSFERFEIFPKNSSWPNHTTNGVTAACSHEGKSSFYRNLLWLTEKEGSYPNLTNNYTNKKGKEVLVLWGIHHPPNSKEQQNLYQNENAYVFVGTSNYDRIFTPIIAERPKVRDQAGRMNYYWTLLKPGDTIIFEANGNLIAPMYAFALRRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 100 - protein, 346 aa, synthetic construct
IAPLQLGNCSVAGWILGNPECNLSINASSWSYIVETPNPENGTCFPGYFADYEELRCQLSSVSSFERFEIFPKNSSWPNHTVTGVSASCSHNGKSSFYRNLLWLTGKNGLYPNLSKNYTNNKSKEVLVLWGVHHPPNIGNQRALYHTENAYVLVVSSHYDRVFTPIIAKRPKVRDQEGRINYYWTLLEPGDTIIFEANGNLIAPWYAFALSRGFGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 101 - protein, 347 aa, synthetic construct
VAPLHLGNCSIAGWILGNPECESLNTSSNWSYIVETSNSDNGTCFPGNFINYEELRCQLSSVSSFERFEIFPKNSSWPNHDSNKGVTAACPHAGAKSFYKNLIWLVKKGNSYPKLNQSYINDKGKEVLVLWGIHHPSTTADQQSLYQNEDTYVFVSTSRYDKVFKPIIATRPKVRDQEGRMNYYWTLVEPGDKITFEATGNLVVPRYAFTMERNAGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 102 - protein, 374 aa, synthetic construct
EKRGKLCPKCLNCTDLDVALGRPKCTGKIPSARTSELHEERPVTSGCFPIRHDLTKIRCLPNLLRGYEHIRLSTHNVINAENAPGGPYKIGTSGSCPNITNGNGFFATMAWAVPKNDKNKTATEPLTIEVPYICTEGEDQITVWGFHSDNETQMAKLYGDSKPQQFTSIANGVTTHYVSQIGGFPNQTEDGGLPQSGRIVVDYMVQKSGKTGIIIYQRGILLPQKVWCASGRSKVIKGSGSGSGECHSPIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 103 - protein, 373 aa, synthetic construct
RTRGKLCPDCLNCTDLDVALGRPECVGTTPSAKTSELHEREPVTSGCFPIRHDLTKIRCLPNLLRGYEKIRLSTQNVIDAEKAPGGPYRLGTSGSCPNATSKIGFFATMAWAVPKDNYKNATEPLTVEVPYICTEGEDQITVWGFHSDNKTQMKSLYGDSKPQQFTSIANGVTTHYVSQIGDFPDQTEDGGLPQSGRIVVDYMMQKPGKTGIIIYQRGVLLPQKVWCASGRSKVTKGSGSGSGECHSPIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 104 - protein, 338 aa, synthetic construct
KQLDGEDCTLIDALLGDPHCDGFQNETWDQFTERSKAYSNCYPYDVPDYESLRCLVAESGTLEFITEGFTWTGVTQNGGSNACKRNGESGFFSRLNWLTKSGSTYPVLNVTMPNNDNFDKLYIWGVHHPSTNQEQKSLYVQESGRVTVSTRRSQQTINPNIGSRPWVRGLSSRISIYYTIVKPGDVLTINSNGNLIAPRGYFKMKTGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 105 - protein, 347 aa, synthetic construct
VKPLQLRDCSVAGWLLGNPECDEFINVSEWSYIVEKENPTNDLCFPGSFNDYEELKCLLSAINHFEKIQIIPKSSWSDHEASSGVSSACPYLGSPSFFRNVVWLIKKNSTYPTIKKSYNNTNQEDLLVLWGIHHPNDAAEQTRLYQNPTTYIIIGTSTLDQVLTPVIATRPKVNGQSGRMEFFWTILKPNDAIIFESNGNFIAPEYAYKIVKKGDSGSGSGCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 106 - protein, 346 aa, synthetic construct
IKPLELGDCSIAGWLLGNPECDRLLSVSEWSYITEKENPRDGLCYPGSFNDYEELKCLLSSVKHFEKVKILPKDRWTQHTTTGGSRACAVSGNPSFFRNMVWLTEKGSNYPVAKGSYNNTSGEQMLIIWGVHHPNDETEQRTLYQNVGTYVVVGTSTLDKVSTPIIATRPKVNGQGGRMEFSWTLLDMWDTIIFESTGNLIAPEYGFKISKRGSGSGSGSCIEHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 107 - protein, 370 aa, synthetic construct
IAPLQLGNCSVAGWILGNPECELLISKESWSYIVETPNPENGTCFPGYFADYEELRCQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRVFTPIIAKRPKVRDQEGRINYYWTLLEPGDTIIFEANGNLIAPWYAFALSRGFGSGSGSCIENINSKIYHIENEIARIKKLIGESGGSGGESAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 108 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYDRVFTPIIVKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 109 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYSREFTPVIVKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 110 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVVVVSSHYDRRFTPQIVKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 111 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVLVVSSHYDRRFTPQIAKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 112 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVAVVSSHYSRVFTPIILKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 113 - protein, 353 aa, synthetic construct
IAPLQLGNCSVAGWILGNPECELLISKESWSYIVETPNPENGTCFPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVAVVSSHYDRVFTPTILKRPKVRDQEGRINYYWTLLEPGDTIIFEANGNLIAPWYAFALSRGFGSGSGSCIENINSKIYHIENEIAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 114 - protein, 118 aa, synthetic construct
IAELAYLLGELAYKLGEYRIAIRAYRIALKSDPNNAEAWYNLGNAYYKQGRYREAIEYYQKALELDPNNAEAWYNLGNAYYERGEYEEAIEYYRKALRLDPNNADAMQNLLNAKMREE

(19) United States
(12) Patent Application Publication
(10) Pub. No.: US 2025/0127877 A1
(43) Pub. Date: Apr. 24, 2025

(54) NOVEL IMMUNOGENS FOR INFLUENZA VIRUS VACCINES

(71) Applicants: UNIVERSITY OF WASHINGTON, Seattle, WA (US); THE USA, as represented by THE SECRETARY, DEPARTMENT OF HEALTH AND HUMAN SERVICES, Bethesda, MD (US)

(72) Inventors: Neil P. KING, Seattle, WA (US); Daniel ELLIS, Seattle, WA (US); Anne DOSEY, Seattle, WA (US); Masaru KANEKIYO, Seattle, WA (US)

(21) Appl. No.: 18/723,029

(22) PCT Filed: Jan. 5, 2023
(86) PCT No.: PCT/US2023/060130

§ 371 Date: Jun. 21, 2024

Related U.S. Application Data
(60) Provisional application No. 63/297,523, filed on Jan. 7, 2022.

(51) Int. Cl. A61K 39/145; A61K 39/00; A61P 31/16; C07K 14/005; C12N 7/00

(52) U.S. Cl. CPC A61K 39/145; A61P 31/16; C07K 14/005; C12N 7/00; A61K 2039/55555; C12N 2760/16122; C12N 2760/16134

(57) ABSTRACT

Disclosed herein are polypeptides that include (a) a heptad motif domain comprising an amino acid sequence according to the genus (I-X1-X2-I-X3-X4-X5)ₙ, wherein X1, X2, X3, X4, and X5 may independently be any amino acid other than proline, and wherein n can be 1-30; and (b) a second domain selected from the group consisting of (i) a polypeptide antigen, and (ii) a polypeptide component of a nanoparticle, nanoparticles including such polypeptides, and uses thereof.

### CROSS REFERENCE

This application claims priority to U.S. Provisional Patent Application Ser. No. 63/297,523 filed Jan. 7, 2022, incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on Jan. 3, 2023 having the file name “21-1540-WO.xml” and is 121 kb in size.

### BACKGROUND

Influenza vaccines maintain massive biomedical importance, both for the clinical prevention of disease and the general study of vaccinology. The influenza hemagglutinin (HA) glycoprotein is notably relevant, which is a homotrimeric class I fusion protein responsible for both receptor binding to sialic acids and cell entry. The head contains an upper receptor binding domain (RBD) which presents an apical receptor binding site (RBS) and a lower “vestigial esterase” subdomain, while the stem domain holds metastable membrane fusion machinery. The HA head is the predominant target of antibodies elicited by existing commercial influenza vaccines. However, the head domain of HA is characteristic for both possessing a combination of hypervariability and immunodominance. This combination of properties is considered largely responsible for current vaccines eliciting antibody responses that are narrow in specificity, which necessitates regular updates to vaccine formulations when circulating viruses mutate.

### SUMMARY

In one aspect, the disclosure provides polypeptides, comprising:
(a) a first domain comprising a heptad motif comprising an amino acid sequence according to the genus (I-X1-X2-I-X3-X4-X5)ₙ, wherein X1, X2, X3, X4, and X5 may independently be any amino acid other than proline, and wherein n can be 1-30; and
(b) a second domain selected from the group consisting of:
   (i) a polypeptide antigen, and
   (ii) a polypeptide component of a nanoparticle.

In various embodiments, the first domain comprises the amino acid sequence selected from the group consisting of SEQ ID NO:6-27, or selected from the group consisting of SEQ ID NO: 6 and 8-27, or selected from the group consisting of SEQ ID NO:6, 8-25, and 27, or selected from the group consisting of SEQ ID NO:6 and 13. In another embodiment, the second domain comprises a polypeptide antigen. In some embodiments, the polypeptide antigen is selected from the group including but not limited to influenza antigens, coronavirus antigens, human immunodeficiency antigens, cytomegalovirus antigens, respiratory syncytial virus antigens, metapneumovirus antigens, parainfluenza virus antigens, Ebola virus antigens, Lassa virus antigens, and Nipah virus antigens, or immunogenic portions thereof. In further embodiments, the polypeptide antigen comprises an influenza hemagglutinin (HA) protein, or immunogenic portion thereof including but not limited to a HA head domain, a HA receptor binding domain (RBD), and/or a HA apical receptor binding site (RBS), or immunogenic portion thereof. In one embodiment, the HA protein or immunogenic portion thereof comprises an amino acid sequence at least 75%, 80%, 85%, 90%, 91%, 92%, 93%, 94%, 95%, 96%, 97%, 98%, or 99% identical to the amino acid sequence of SEQ ID NO: 1 residues 51-264 (the head domain, bolded in SEQ ID NO:1), wherein the HA protein or immunogenic portion thereof includes 1, 2, 3, 4, or all S of the following amino acid residues relative to SEQ ID NO:1 when aligned by protocol 1 or protocol 2: 107C, 203L, 210D, 212V or I (or 212V), and/or 216I, wherein residues in parentheses are not present in mature HA protein.

In another embodiment, the second domain comprises a polypeptide component of a nanoparticle. In one embodiment, the polypeptide component of a nanoparticle comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:114.

In a further embodiment, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:83-113.

In another aspect, the disclosure provides mutated HA polypeptide comprising an amino acid sequence at least 75%, 80%, 85%, 90%, 91%, 92%, 93%, 94%, 95%, 96%, 97%, 98%, or 99% identical to the amino acid sequence of SEQ ID NO:1, wherein the HA protein or immunogenic portion thereof includes 1, 2, 3, 4, or all 5 of the following amino acid residues relative to SEQ ID NO: 1 residues 51-264 (the head domain, bolded in SEQ ID NO: 1) when aligned by protocol 1 or protocol 2: 107C, 203L, 210D, 212V or I (or 212V), and/or 216I. In some embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 33-39 and 42-52, wherein at least 1, 2, 3, 4, 5, 6, 7, 8, 9, 10, 11, 12, 13, 14, or 15 of the bold-faced residue(s) is/are present in the polypeptide. In another embodiment, the disclosure provides mutated HA polypeptides comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:29-32 and 53-57, wherein mutations relative to wild type are noted in bold, and wherein the at least 1, 2, 3, 4, 5, 6, 7, or all of the mutations are present in the polypeptide.

In other embodiments, the disclosure provides nucleic acid encoding the polypeptide of any embodiment of the disclosure, expression vectors comprising the nucleic acid of any embodiment herein operatively linked to a suitable control sequence, and host cells comprising a polypeptide, nucleic acid, and/or expression vector of any embodiment.

In another embodiment, the disclosure comprises nanoparticles comprising a plurality of the polypeptides of any embodiment of the disclosure. In one embodiment, the panoparticle comprises: (a) a plurality of first assemblies, each first assembly comprising a plurality of identical first polypeptides, wherein the first polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence selected from the group consisting of SEQ ID NOS: 2-4, where residues in parentheses are optional and may be present or absent; and (b) second polypeptides, wherein the second polypeptides comprise the polypeptide of any embodiment herein that includes a third domain such that the polypeptide comprises both a polypeptide antigen and a polypeptide component of a nanoparticle, wherein the polypeptide component of a nanoparticle comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:114; wherein the plurality of first assemblies non-covalently interact with the plurality of second assemblies to form a nanostructure; and wherein the nanostructure displays multiple copies of one or more immunogenic polypeptide antigens, on an exterior of the nanostructure. In one embodiment, the second polypeptides comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 58-79, or SEQ ID NO:58 and 60-79, or SEQ ID NO: 58 and 65. In another embodiment, the second polypeptides comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 83-113.

In one aspect, the disclosure provides pharmaceutical compositions comprising (a) the polypeptide, nucleic acid, recombinant expression vector, cell, and/or nanoparticle of any embodiment; and (b) a pharmaceutically acceptable carrier. In another aspect, the disclosure provides vaccines comprising the polypeptide, nucleic acid, recombinant expression vector, cell, nanoparticle, or composition of any embodiment herein. In further aspects, the disclosure provides methods to vaccinate a subject against an infectious agent, including but not limited to the influenza virus, the method comprising administering to the subject the polypeptide, nucleic acid, recombinant expression vector, cell, nanoparticle, or composition of any embodiment herein. In further embodiments, the disclosure provides method of detecting anti-infectious agent antibodies, comprising contacting at least a portion of a sample being tested for the presence of anti-infectious agent antibodies, such as influenza antibodies, with a polypeptide or nanoparticle of any embodiment; and detecting the presence of an antibody-nanoparticle complex, wherein the presence of an antibody-nanoparticle complex indicates that the sample contains anti-infectious agent antibodies, such as anti-influenza antibodies.

In some aspects, the disclosure provides method to identify a subject having anti-infectious agent antibodies, including but not limited to anti-influenza virus antibodies, comprising contacting a sample from a subject being tested with a polypeptide or nanoparticle of any embodiment herein; and, analyzing the contacted sample for the presence of an antibody-nanoparticle complex, wherein the presence of an antibody-nanoparticle complex indicates the subject has anti-infectious agent antibodies, such as anti-influenza antibodies. In other aspects, the disclosure provides methods to identify a subject that has been exposed to an infectious agent, including but not limited to an influenza virus, the method comprising contacting at least a portion of a sample from a subject being tested with a polypeptide or nanoparticle of any embodiment herein, analyzing the contacted sample for the presence or level of an antibody/nanoparticle complex, wherein the presence or level of antibody-nanoparticle complex indicates the presence or level of recent anti-infectious agent antibodies, such as anti-influenza antibodies; and comparing the recent antibody level with a past antibody level; wherein an increase in the recent antibody level over the past antibody level indicates the subject has been exposed to infectious agent, such as influenza virus subsequent to determination of the past antibody level.

In other embodiments, the disclosure provides methods for measuring the response of a subject to a vaccine, the method comprising: administering to the subject a vaccine for an infectious agent, including but not limited to influenza virus; contacting at least a portion of a sample from the subject with the polypeptide or nanoparticle of any embodiment herein; and analyzing the contacted sample for the presence or level of an antibody/nanoparticle complex, wherein the presence or level of antibody-nanoparticle complex indicates the presence or level of recent anti-infectious agent antibodies; wherein an increase in the level of antibody in the sample over the pre-vaccination level of antibody in the subject indicates the vaccine induced an immune response in the subject.

### DESCRIPTION OF THE FIGURES

FIG. 1. Design and characterization of nanoparticle components rigidly displaying closed trimeric HA heads (“TriHead”). A) Design model of the initial successful TriHead design, SEQ ID NO:95, which features a rigid helical extension from the nanoparticle N-terminus, SEQ ID NO:13, a designed linker to connect to the HA head, a disulfide between the head and the N-terminal nanoparticle extension, and a hydrophobic interface designed between the individual head domains. B) Fractional binding of anti-HA antibodies to the final TriHead design (“Closed trihead”, SEQ ID NO:95), the original design lacking the designed disulfide and head-head interface (“open native head”), SEQ ID NO:83, and trimeric HA ectodomain fused to foldon (“Ectodomain HA control”, SEQ ID NO:1) as measured by BLI. The D2 H1-1/H3-1 antibody recognizes an epitope at the head-head interface and is not neutralizing, while C05 recognizes the highly conserved receptor binding site. C) 2D class averages of the initial TriHead design (SEQ ID NO:95) after assembled as a nanoparticle, obtained by cryo-EM.

FIG. 2. A) Design models of TH-A/South Carolina/1/1918 (TH-SC18) (SEQ ID NO: 92, 98), TH-A/Puerto Rico/8.1943 (TH-PR34), (SEQ ID NO: 93, 99) TH-A/New Caledonia/20/1999 (TH-NC99), SEQ ID NO: 94-96, 100, and TH-A/Michigan/45/2015 (TH-MI15) (SEQ ID NO: 97, 101 that show the TriHead hydrophobic head stabilizing mutations at the trimeric interface as labeled and as side-chain stick representations. B) Melting temperatures (Tm) of TriHeads with the designed hydrophobic bead interface, as well as added glycosylation sites on the side of the HA head that all show higher Tms than their counterparts with only the designed disulfide bond lacking the designed interface, showing increased stability with added interface mutations. The hyperglycosylated TriHeads are TH-SC18+5 glycans, SEQ ID NO: 98, TH-PR34+7 glycans, SEQ ID NO: 99, TH-NC99+5 glycans, SEQ ID NO: 100, TH-MI15+4 glycans, SEQ ID NO: 101. The hyperglycosylated ‘Disulfide only’ constructs are SC18+5 glycans, SEQ ID NO: 88, PR34+7 glycans, SEQ ID NO: 89, NC99+5 glycans, SEQ ID NO: 90, MI15+4 glycans, SEQ ID NO: 91.

FIG. 3. Biolayer interferometry (BLI) data showing high binding of known anti-HA RBS monoclonal antibodies (mAbs) 5J8, anti-PR34, and C05 to both A) disulfide only HA heads of SEQ ID NOs: 84-87 and B) TriHeads of SEQ ID NOs: 92, 93, 94, 97, indicative that the RBS is antigenically intact and available for mAb binding. In contrast, anti-head trimerization interface mAb FluA20 showed C) high binding to all disulfide only heads but D) minimal binding to all TriHeads, indicative that the designed trimeric head interface is necessary to close the HA heads together and prevent FluA20 binding.

FIG. 4. Negative stain electron microscopy (nsEM) 2D class averages of A) TriHeads of SEQ ID NOs: 92, 93, 94, 97, where HA beads are resolved as displayed on the surface of the I53_dn5 nanoparticle. This is in contrast to the B) Disulfide only PR34 class averages, SEQ ID NO: 85, where HA heads are not resolved. These class averages show that the designed interface helps stabilize and rigidify the HA heads.

FIG. 5. Mutations that enhance different strains of TriHead expression or stabilize HA head interface closure. A) List of expression enhancing mutations and head interface closure mutations for the various TriHead constructs listed, all verified using SDS-PAGE and SEC for expression (data not shown). B) nsEM 2D class average of the influenza B TriHead (SEQ ID NO: 103) as displayed on the I53_dn5 nanoparticle (top right panel). C) BLI data of the H3 TriHead (SEQ ID NO: 104) where C05 binds strongly and FluA20 binds only minorly, showing it is antigenically intact.

FIG. 6. Hypervariable mutations in the RBS periphery listed in the Tables 2 and 4 are contained in constructs shown on an SDS-PAGE gel showing that these were all able to be expressed. These were designed as an immune refocusing tool, with the intention for all 20 TriHead variants to be co-displayed on a I53_dn5 nanoparticle, and thus deter strain-specific responses against the poorly conserved RBS periphery and simultaneously boost responses against the highly conserved RBS.

FIG. 7. Structural and immunogenic characterization of immunogens displaying bead domains in different states of closure and/or geometric spacings, SEQ ID NO: 83, 94-96, 107. A) 3D class averages of five HA-head based nanoparticle immunogens obtained by negative stain EM or cryo-EM. Structural characterization of the Closed/Flexible and Open/Flexible constructs could not resolve the head antigens, and head antigens shown for these constructs are representative cartoons to demonstrate flexibility. Models of nanoparticles and antigens are all rigid body fits. B) Immunization schedule in mice, with immunizations on weeks 0, 4 and 8 with serum collection on week 10. All immunizations contained equimolar amounts of the HA head, with all doses near 1.5 ug. C) Week 10 ELISA endpoint binding titers against HAs from diverse H1N1 strains, including strain-matched NC99 HA, and mismatched Malaysia54 and USSR77 HAs. D) Week 10 strain-matched microneutralization titers against H1N1 NC99 virus. Data plotted are titers for which 80% of virus entry to host cells was inhibited.

### DETAILED DESCRIPTION

All references cited are herein incorporated by reference in their entirety.

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Any N-terminal methionine residues are optional and may be present or absent. In all embodiments, the polypeptides may optionally include N- and/or C-terminal deletions of 1, 2, 3, 4, or 5 residues relative to the recited sequence.

In a first aspect, the disclosure provides polypeptides, comprising:
(a) a first domain comprising a heptad motif comprising an amino acid sequence according to the genus (I-X1-X2-I-X3-X4-X5)ₙ, wherein X1, X2, X3, X4, and X5 may independently be any amino acid other than proline, as all amino acids except proline can comprise alpha helical secondary structure, and wherein n can be 1-30; and
(b) a second domain selected from the group consisting of:
   (i) a polypeptide antigen, and
   (ii) a polypeptide component of a nanoparticle.

The polypeptides of this aspect comprise a heptad motif that is shown in the examples below to mediate a connection between antigen (exemplified by hemagglutinin (HA) head domains) and a protein nanoparticle that permits control over both the local oligomeric state of antigens to be either monomeric or trimeric, and their precise rigid spatial organization on a nanoparticle. The X residues are solvent exposed and thus modifiable, while the I residues are buried. Alterations to both features allows for tuned immunogenicity of different epitopes on the antigen.

The second domain may be directly linked to the polypeptide or may be linked via any suitable polypeptide linker, including but not limited to a flexible linker.

The first domain comprises at least 1 heptad motif of the genus I-X1-X2-I-X3-X4-X5. The first domain may comprise 1-30 such heptad motifs. This heptad motif can arbitrarily start or end at any residue, so long as the sequence I-X1-X2-I-X3-X4-X5 is present in the first domain. In one embodiment, each heptad repeat is identical. In another embodiment, the heptad repeats are not all identical. In some embodiments, each heptad repeat is different.

In one embodiment, X1 is selected from the group consisting of A, D, E, H, K, N, Q, R, S, Y, and T; or X1 is selected from the group consisting of E, Y, A, and R.

In another embodiment, X2 is selected from the group consisting of A, D, E, H, K, N, Q, R. S, and T; or wherein X2 is selected from the group consisting of E, H, R, and N.

In a further embodiment, X3 is selected from the group consisting of A, D, E, H, K, L, N, Q, R, S, and T; or wherein X3 is selected from the group consisting of L, E, K, and N.

In one embodiment, X4 is selected from the group consisting of A, D, E, H, K, N, Q, R, S, and T; or wherein X4 is selected from the group consisting of S, D, N, and K.

In another embodiment, X5 is selected from the group consisting of A, D, E, H, K, L, N, Q, R, S, and T; or wherein X5 is selected from the group consisting of K, E, and L. These residues are preferred due to their propensity to form alpha helical secondary structure and because these positions are at the boundary or surface of the protein and so more hydrophilic residues are preferred at such positions, so most of these residues (all except A and L) are hydrophilic.

In a further embodiment, the first domain comprises the amino acid sequence selected from the group consisting of SEQ ID NO:6-27. In some embodiments, the first domain comprises amino acid sequence selected from the group consisting of SEQ ID NO:6 and 8-27. In other embodiments, the first domain comprises amino acid sequence selected from the group consisting of SEQ ID NO:6, 8-25, and 27. In further embodiments, first domain comprises amino acid sequence selected from the group consisting of SEQ ID NO:6 and 13. The sequence of these heptad motifs is provided in Table 1.

TABLE 1
Exemplary heptad motifs

| SEQ | | |
|---|---|---|
| ID | | |
| NO | Name | Amino acid sequence |
| 6 | ext-1-heptad-A_dn5b | IEHIENE |
| 7 | ext-2-heptad-A_dn5b | IEDKIEEILSKIYHIENE |
| 8 | ext-2-heptad-B_dn5b | NIENKIEEIEKKIYHIENE |
| 9 | ext-2-heptad-C_dn5b | EIEKKIEEIEKKIERIENE |
| 10 | ext-2-heptad-D_dn5b | NINKKIEEIEKKIYHIENE |
| 11 | ext-2-heptad-E_dn5b | INKKIEEIEKKIYHIENE |
| 12 | ext-2-heptad-F_dn5b | EEINKKIEEIEKKIYHIENE |
| 13 | ext-2-heptad-G_dn5b | IENINSKIYHIENE |
| 14 | ext-2-heptad-H_dn5b | IEAILSKIYHIENE |
| 15 | ext-2-heptad-I_dn5b | IENIASKIYHIENE |
| 16 | ext-2-heptad-J_dn5b | IEqILRKIYHIENE |
| 17 | ext-3-heptad-A_dn5b | IEDKIEEILRKIEEIEKKIYHIENE |
| 18 | ext-3-heptad-B_dn5b | EEIKDKIEEILRKIEEIEKKIYHIENE |
| 19 | ext-3-heptad-C_dn5b | ILSKIYHIERKIEEILNE |
| 20 | ext-3-heptad-D_dn5b | IEDKIYHIERKIEEILNE |
| 21 | ext-3-heptad-E_dn5b | NIEDKIYHIERKIEEILNE |
| 22 | ext-3-heptad-F_dn5b | LSKIYHIERKIEEILNE |
| 23 | ext-6-heptad-A_dn5b | ILSKIYHIEDKIEEINRKIEHILSKIYHIERKIEEILNE |
| 24 | ext-6-heptad-B_dn5b | IEDKIYHIEDKIEEINRKIEHILSKIYHIERKIEEILNE |
| 25 | ext-6-heptad-C_dn5b | NIEDKIYHIEDKIEEINRKIEHILSKIYHIERKIEEILNE |
| 26 | ext-6-heptad-D_dn5b | IENINSKIYHIEDKIEEINRKIEHILSKIYHIERKIEEILNE |
| 27 | ext-7-heptad-A_dn5b | IEDKIEEILSKIYHIEDKIEEINRKIEHILSKIYHIERKIEEILNE |

In one embodiment, the second domain comprises a polypeptide antigen. This embodiment may be used, for example, as an immunogen or vaccine to produce an immune response against the polypeptide antigen. Any antigen may be used, as appropriate for an intended purpose, including, but not limited to the pathogen-specific antigens or immuonogenic portions thereof such as antigens from influenza viruses, hepatitis (A, B, C, E, etc.) virus, human papillomavirus, herpes simplex viruses, cytomegalovirus, Epstein-Barr virus, rhinovirus, enterovirus, measles virus, mumps virus, polio virus, rabies virus, human immunodeficiency virus, respiratory syncytial virus, Rotavirus, rubella virus, varicella zoster virus, Ebola virus, cytomegalovirus, Marburg virus, norovirus, variola virus, any Flavivirus including but not limited to West Nile virus, yellow fever virus, dengue virus, tick-borne encephalitis virus, zika virus, and Japanese encephalitis virus, Bacillus anthracis, Bordetalla pertussis, Chlamydia trachomatis, Clostridium tetani, Clostridium difficile, Corynebacterium diptheriae, Coxiella burnetii, Escherichia coli, Haemophilus influenza, Helicobacter pylori, Leishmania donovani, L. tropica and L. braziliensis, Mycobacterium tuberculosis, Mycobacterium leprae, Neisseria meningitis, Plasmodium falciparum, P. ovale, P. malariae and P. vivax, Pseudomonas aeruginosa, Salmonella typhi, Schistosoma hematobium, S. mansoni, Streptococcus pneumoniae (group A and B), Staphylococcus aureus, Toxoplasma gondii, Trypanosoma brucei, T. cruzi and Vibrio cholerae; antigens expressed in or on the surface of tumors/tumor cells (including but not limited to p53 (colorectal cancer), alphafetoprotein (germ cell tumors; hepatocellular carcinoma), carcinoembryonic antigen (bowel cancers), CA-125 (ovarian cancer), human epidermal growth factor receptor-2 (HER-2, breast cancer), MUC-1 (breast cancer), NY-ESO-1 (esophageal cancer, non-small-cell lung cancer), epithelial tumor antigen (breast cancer), tyrosinase (malignant melanoma), Disialoganglioside (GD2, neuroblastoma), melanoma-associated antigen gene-1 (MAGE-1 (malignant melanoma), and beta amyloid (for Alzheimer's and other amyloid-based diseases), etc.

In various non-limiting embodiments, the polypeptide antigen may be selected from the group including but not limited to influenza antigens, coronavirus antigens, human immunodeficiency antigens, cytomegalovirus antigens, respiratory syncytial virus antigens, metapneumovirus antigens, parainfluenza virus antigens, Ebola virus antigens, Lassa virus antigens, and Nipah virus antigens, or immunogenic portions thereof.

In one specific embodiment, the polypeptide antigen comprises an influenza hemagglutinin (HA) protein, or immunogenic portion thereof. Any immunogenic portion of an influenza HA protein may be used, including but not limited to a HA head domain, a HA receptor binding domain (RBD), and/or a HA apical receptor binding site (RBS), or immunogenic portion thereof. In one embodiment, the HA protein or immunogenic portion thereof are selected from the group consisting influenza A and influenza B HA proteins, or immunogenic portions thereof. In some embodiments, the HA protein is selected from the group consisting of immunogenic portions of a HA protein from strains H1, H2, H3, H4, H5, H6, H7, H8, H9, H10, H11, H12, H13, H14, H15, H16 H17, and H18 HA protein, or immunogenic portions thereof. In other embodiments, the HA proteins comprise immunogenic portions of HA proteins from strains including but not limited to H10N4, H10N5, H10N7, H10N8, H10N9, H11N1, H11N3, H11N2, H11N4, H11N6, H11N8, H11N9, H12N1, H12N4, H12N5, H12N8, H13N2, H13N3, H13N6, H13N7, H14N5, H14N6, H15N8, H15N9, H16N3, H1N1, H1N2, H1N3, H1N6, H1N9, H2N1, H2N2, H2N3, H2N5, H2N7, H2N8, H2N9, H3N1, H3N2, H3N3, H3N4, H3N5, H3N6, H3N8, H3N9, H4N1, H4N2, H4N3, H4N4, H4N5, H4N6, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N4, H8N5, H9N1, H9N2, H9N3, H9N5, H9N6, H9N7, H9N8, and H9N9. In other embodiments, the immunogenic portion of the HA proteins comprise an immunogenic portion of n HA protein from one or more strains selected from (a) A/Michigan/45/2015 (H1N1), (b) A/Hong Hong/4801/2014 (H3N2), (c) B/Brisbane/60/2008 (Victoria lineage), and (d) B/Phuket/3073/2013 (Yamagata lineage), (d) A/Idaho/07/2018 (H1N1) pdm09-like virus, (e) A/Perth/1008/2019 (H3N2)-like virus (updated), (f) B/Colorado/06/2017-like (Victoria lineage) virus (updated), (g) B/Phuket/3073/2013-like (Yamagata lineage) virus, (h) A/Brisbane/02/2018 (H1N1) pdm09-like, (i) A/South Australia/34/2019 (H3N2)-like, (j) B/Washington/02/2019-like (Victoria lineage), and (k) B/Phuket/3073/2013-like (Yamagata lineage).

In certain non-limiting embodiments, the HA protein or immunogenic portion thereof comprises an amino acid sequence at least 75%, 80%, 85%, 90%, 91%, 92%, 93%, 94%, 95%, 96%, 97%, 98%, or 99% identical to the amino acid sequence of SEQ ID NO: 1 residues 51-264 (the head domain, bolded in SEQ ID NO:1), wherein the HA protein or immunogenic portion thereof includes 1, 2, 3, 4, or all 5 of the following amino acid residues relative to SEQ ID NO: 1 when aligned by protocol 1 or protocol 2: 107C, 203L, 210D, 212V/I (or 212V), and/or 216I, wherein residues in parentheses are not present in mature HA protein and thus are optional and may be included or deleted.

(ATYA)DTICIGYHANNSTDTVDTVLEKNVTVTHSVNLLEDSHNGKLCLLKGIAPLQLGNCSVAGWILGNPECEL
LISKESWSYIVETPNPENGTCYPGYFADYEELREQLSSVSSFERFEIFPKESSWPNHTVTGVSASCSHNGKSSFY
RNLLWLTGKNGLYPNLSKSYVNNKEKEVLVLWGVHHPPNIGNQRALYHTENAYVSVVSSHYSRRFTPEIAKRPKV
RDQEGRINYYWTLLEPGDTIIFEANGNLIAPWYAFALSRGFGSGIITSNAPMDECDAKCQTPQGAINSSLPPQNV
HPVTIGECPKYVRSAKLRMVTGLRNIPSIQSRGLFGAIAGFIEGGWTGMVDGWYGYHHQNEQGSGYAADQKSTQN
AINGITNKVNSVIEKMNTQFTAVGKEFNKLEPPMENLNKKVDDGFLDIWTYNAELLVLLENERTLDFHDSNVKNL
YEKVKSQLKNNAKEIGNGCFEFYHKCNNECMESVKNGTYDYPKYSEESKLNREKIDGV (SEQ ID NO: 1;
ectodomain of HA sequence from H1 NC99)

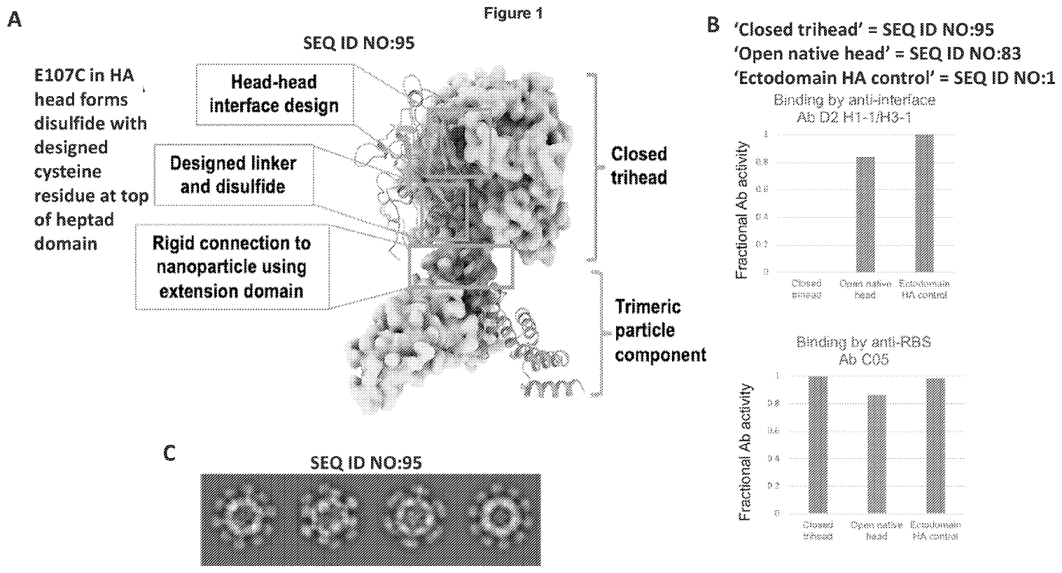

Each of these mutations (107C, 203L, 210D, 212V/I, and/or 216I) is shown in the accompanying examples to stabilize trimer formation. See, for example, FIG. 1B and discussion in the examples.

As used throughout this application, “Protocol 1” and “Protocol 2” both permit alignment of polypeptide against the reference sequence, taking insertions and deletions into account. Thus, the percent identity requirement is based on alignment with the reference sequence while discounting insertions or deletions relative to the reference polypeptide.

### Protocol 1

1. To run a BLASTp alignment online, use the National Center for Biotechnology Information (NCBI) server (or see the article www.ncbi.nlm.nih.gov/pmc/articles/PMC146917/).
   a. blast.ncbi.nlm.nih.gov/Blast.cgi?PAGE=Proteins
2. Set up a BLAST alignment using the following settings:
   Use the option for “Align two or more sequences”
   Enter the subtype-specific reference strain sequence for the relevant NA subtype into the “Enter Query Sequence” section
   Enter any sequence of the same NA subtype into the “Enter Subject Sequence” section
   Algorithm: blastp (protein-protein BLAST)
   Expect threshold: 0.1
   Word size: 6
   Max matches in a query range: 0
   Matrix: BLOSUM62
   Gap costs:
      Existence: 11
      Extension: 1
   Filter low complexity regions?: No
   Mask:
      For lookup table only?: No
      Lower case letters?: No
3. Run the analysis by clicking the “BLAST” button
4. Click on the “Alignments” tab to show the alignment between the two sequences
5. For each sequence position of interest, identify the number according to the “Query” sequence. Then identify the corresponding residue position in the “Sbjet” sequence that has been aligned with the position of the “Query” sequence,

### Protocol 2

1. To run a protein BLASTp alignment on a personal computer or server, install BLAST for command line execution or identify a computer or server that already has it installed.
   a. Directions for installation can be found in the user manual:
      “BLAST® Command Line Applications User Manual”, National Center for Biotechnology Information (US). Bethesda, MD. 2008. www.ncbi.nlm.nih.gov/books/NBK279690/
2. Generate a file in FASTA format that contains the desired subtype-specific reference strain for the relevant NA subtype. In the command below, this file will be named “query.fasta”.
3. Generate a second file in FASTA format that contains an NA sequence of interest from the same subtype. In the command below, this file will be named “sbjct.fasta”
4. Execute the following command using a program such as Terminal, iTerm2, Windows Console, Linux console or other similar terminal emulators. This will generate results in a file named “results.txt”.
   blastp-query query.fasta-subject sbjct.fasta-matrix BLOSUM62-evalue 0.1-word_size 6-gapopen 11-gapextend 1-out results.txt
5. Open results.txt and view the section showing alignment of the two sequences. For each sequence position of interest, identify the number according to the “Query” sequence. Then identify the corresponding residue position in the “Sbjet” sequence that has been aligned with the position of the “Query” sequence.

In another embodiment, the HA protein or immunogenic portion thereof further includes 1, 2, 3, 4, 5, 6, 7, 8, 9, 10, or all 11 of the following amino acid residues, residue numbering relative to SEQ ID NO: 1: 58N, 101N, 126N, 203I/F/L/V/A, 205A or 205G, 212E, 214T, 216L/V/Q/T, 218V or 218L, 221P, and/or 244I. Each of these mutations stabilizes trimer formation and/or boost protein expression.

In a further embodiment, the HA protein or immunogenic portion thereof includes a combination of amino acid residues selected from the group consisting of, residue numbering relative to SEQ ID NO:1;
126N/203I/205A/210D/212V or I (or 212V)/216L,
58N/203F/205G/210D/212V or I (or 212V)/216I,
203L/210D/212V or I (or 212V)/216I,
203V/210D/212V or I (or 212V)/216I/218V,
203V/212E/216V/218V,
203V/210D/216Q/218V,
203L/210D/216Q.
203A/212V or I (or 212V)/216I/218L,
203A/210D/212V or I (or 212V)/216T/218L,
101N/210D/212V or I (or 212V)/216I,
203I/210D/212V or I (or 212V)/214T/216V/221P/244I, and
203V/210D/212V or I (or 212V)/216I/244I.

In another embodiment, the HA protein or immunogenic portion thereof further includes 1, 2, 3, 4, 5, 6, 7, 8, 9, 10, 11, 12, or all 13 of the following amino acid residues, residue numbering relative to SEQ ID NO: 1: 62E, 70E, 76S, 78G, 78S, 84E, 142N, 143G, 144E, 192K, 198E, 200T, and/or 205S. Each of these mutations is shown in the examples to enhance antigen expression.

In a further embodiment, the HA protein or immunogenic portion thereof includes a combination of amino acid residues selected from the group consisting of the following, residue numbering relative to SEQ ID NO:1:
198E,
76S/84E/198E,
198E/200T/205S,
62E/78G/142N/143G/144E/192K, and/or
70E/78S.

Each of these combinations of mutations enhances antigen expression.

In one embodiment, the HA protein or immunogenic portion thereof further includes a combination of amino acid residues selected from the group consisting of the following, residue numbering relative to SEQ ID NO: 1:
63N/65S/81N/125BN/131T/167N/169T,
58N/60S/76N/78S/93T/124N/131T/163N/165T/167N/169T,
72N/74S/76N/78S/124N/167N/169T/173S,
63N/65S/80N/82S/84N/124N, or
63N/65S/81N/125BN/131T/167N/169T.

These mutations add glycosylation sites to the antigens.

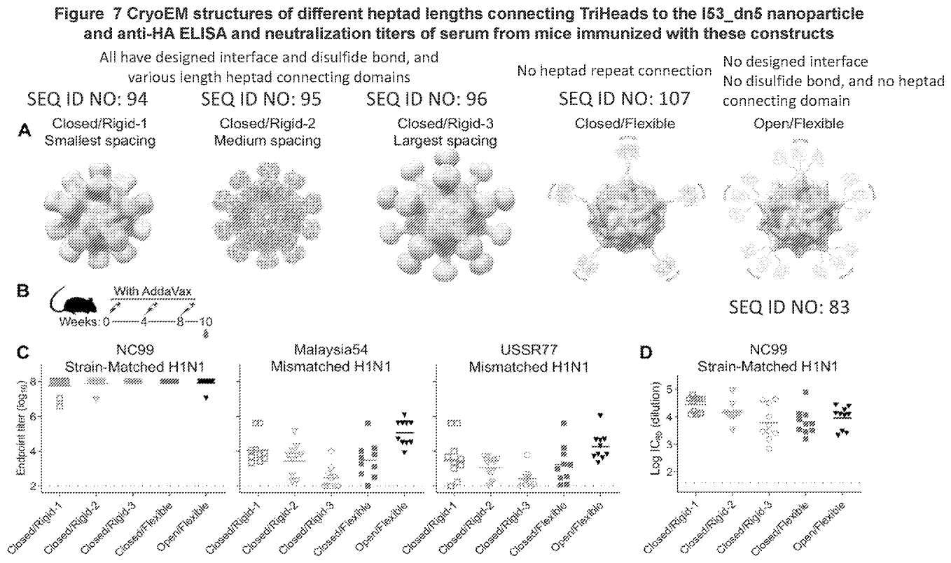

In another embodiment, the HA protein or immunogenic portion thereof comprises Y or F at residue 95 (this mutation allows for release of HA protein when made recombinantly in HEK293 cells, as these cells have HA receptor sialic acid molecules that will bind and tether HA with 95Y, but will release soluble HA with 95F, see Whittle, 2014), residue numbering relative to SEQ ID NO: 1. In a further embodiment, the HA protein or immunogenic portion thereof comprises amino acid residues selected from those listed in Table 2 at the listed positions, residue numbering relative to SEQ ID NO: 1. Also see FIG. 7 and Table 4. These substitutions are hypervariable mutations in the RBS periphery, and are particularly useful, for example, as an immune refocusing tool to deter strain-specific responses against the poorly conserved RBS periphery and simultaneously boost responses against the highly conserved RBS.

TABLE 2

| Position (SEQ ID | |
|---|---|
| NO: 1 numbering) | Residue possibilities |
| 143 | G, Q, S, N |
| 144 | Q, T, E, M, V, K, A |
| 145 | S, K, A, T, L, N |
| 156 | Q, A, S, E, K, G |
| 158 | N, G, E, Q, S, D, K |
| 159 | G, S, N, D, E, T, A |
| 189 | R, N, V, D, S, E, T, K, G, A |
| 192 | Q, R, N, M, E, I, H, A, D |
| 193 | A, S, N, T, G, D |
| 222 | K, Q, L, E, A |
| 225 | G. E, T, D |
| 227 | A, E, S, M, P, Q, H, K |

In one embodiment, the HA protein or immunogenic portion thereof comprises an amino acid sequence at least 75%, 80%, 85%, 90%, 91%, 92%, 93%, 94%, 95%, 96%, 97%, 98%, or 99% identical to the amino acid sequence of SEQ ID NO: 1 or SEQ ID NO:5, wherein the N-terminal methionine residue is optional and may be present or absent.

>NC99 full-length HA
(SEQ ID NO: 5)
(M)KAKLLVLLCTFTATYADTICIGYHANNSTDTVDTVLEKNVTVTHSVNLLEDSANGKLCLLKGIAPLQLGNCS
VAGWILGNPECELLISKESWSYIVETPNPENGTCYPGYFADYEELREQLSSVSSFERFEIFPKESSWPNHTVTGV
SASCSHNGKSSFYRNLLWLTGKNGLYPNLSKSYVNNKEKEVLVLWGVHHPPNIGNQRALYHTENAYVSVVSSHYS
RRFTPEIAKRPKVRDQEGRINYYWILLEPGDTIIFEANGNLIAPWYAFALSRGFGSGIITSNAPMDECDAKCQTP
QGAINSSLPFQNVHPVTIGECPKYVRSAKLRMVTGLRNIPSIQSRGLFGAIAGFIEGGWTGMVDGWYGYHHQNEQ
GSGYAADQKSTQNAINGITNKVNSVIEKMNTQFTAVGKEFNKLERRMENLNKKVDDGELDIWTYNAELLVLLENE
RTLDEHDSNVKNLYEKVKSQLKNNAKEIGNGCFEFYHKCNNECMESVKNGTYDYPKYSEESKLNREKIDGVKLES
MGVYQILAIYSTVASSLVLLVSLGAISFWMCSNGSLQCRICI

SEQ ID NO:5 is the full length NC99 HA protein, and includes the NC99 ectodomain of SEQ ID NO:1, which is shown in bold font in SEQ ID NO:5.

In another embodiment, the HA protein or immunogenic portion thereof comprises an amino acid sequence at least 75%, 80%, 85%, 90%, 91%, 92%, 93%, 94%, 95%, 96%, 97%, 98%, or 99% identical to the amino acid sequence of SEQ ID NO:1, wherein the N-terminal ATYA is optional and may be present or absent.

In other embodiments, the polypeptide antigen comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 28-39 and 42-57, or an immunogenic portion thereof. These are various other HA proteins, as listed above each sequence. Any residue numbering is based on SEQ ID NO:1 residue numbering.

HA Antigen Heads without Inter-Head Stabilizing Mutations

### H1 Beads

>A/New Caledonia/20/1999
(SEQ ID NO: 28)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVSVVSSHYSRRFTPEIAKRPKVRDQEGRINYYWTLLEPGDTIIFEANGNLIAP
WYAFALSRGF

### H1 Disulfide Only Heads

>A/South Carolina/1/1918
(SEQ ID NO: 29)
IAPLQLGKCNIAGWLLGNPECDLLLTASSWSYIVETSNSENGTCYPGDFIDYEELRCQLSSVSSFEKF
EIFPKTSSWPNHETTKGVTAACSYAGASSFYRNLLWLTKKGSSYPKLSKSYVNNKGKEVLVLWGVHHP
PTGTDQQSLYQNADAYVSVGSSKYNRRFTPEIAARPKVRDQAGRMNYYWTLLEPGDTITFEATGNLIA
PWYAFALNR
>A/Puerto Rico/8/1934
(SEQ ID NO: 30)
IAPLQLGKCNIAGWLLGNPECDPLLSVRSWSYIVETPNSENGICYPGDFIDYEELRCQLSSVSSFERF
EIFPKESSWPNHNTNGVTAACSHEGKSSFYRNLLWLTEKEGSYPKLKNSYVNKKGKEVLVLWGIHHPP
NSKEQQNLYQNENAYVSVVTSNYNRRFTPEIAERPKVRDQAGRMNYYWILLKPGDTIIFEANGNLIAP
MYAFALRRGF
>A/New Caledonia/20/1999
(SEQ ID NO: 31)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVEVLWGVHHPP
NIGNQRALYHTENAYVSVVSSHYSRRFTPEIAKRPKVRDQEGRINYYWTLLEPGDTIIFEANGNLIAP
WYAFALSRGF
>A/Michigan/45/2015
(SEQ ID NO: 32)
VAPLHLGKCNIAGWILGNPECESLSTASSWSYIVETSNSDNGTCEPGDFINYEELRCQLSSVSSFERF
EIFPKTSSWPNHDSNKGVTAACPHAGAKSFYKNLIWLVKKGNSYPKLNQSYINDKGKEVLVLWGIHHP
STTADQQSLYQNADAYVFVGTSRYSKKFKPEIATRPKVRDQEGRMNYYWTLVEPGDKITFEATGNLVV
PRYAFTMERNA

HA Antigen Heads without Inter-Head Stabilizing Mutations

### Hyperglycosylated H1 Disulfide Only Heads

>A/South Carolina/1/1918_+5 glycans
(63N/65S/81N/125BN/131T/167N/169T)
(SEQ ID NO: 33)
IAPLQLGNCSIAGWLLGNPECDLLLNASSWSYIVETSNSENGTCYPGDFIDYEELRCQLSSVSSFEKF
EIFPKNSSWPNHTTTKGVTAACSYAGASSFYRNLLWLTKKGSSYPKLSKNYTNNKGKEVLVLWGVHHP
PTGTDQQSLYQNADAYVSVGSSKYNRRFTPEIAARPKVRDQAGRMNYYWTLLEPGDTITFEATGNLIA
PWYAFALNR
>A/Puerto Rico/8/1934_+7 glycans
(58N/60S/76N/78S/93T/124N/131T/163N/165T/167N/169T)
(SEQ ID NO: 34)
IAPLQLGNCSIAGWLLGNPECDPLLNVSSWSYIVETPNSENGTCYPGDFIDYEELRCQLSSVSSFERF
EIFPKNSSWPNHTTNGVTAACSHEGKSSFYRNLLWLTEKEGSYPNLTNNYTNKKGKEVLVLWGIHHPP
NSKEQQNLYQNENAYVSVVTSNYNRRFTPEIAERPKVRDQAGRMNYYWTLLKPGDTIIFEANGNLIAP
MYAFALRRGF
>A/New Caledonia/20/1999_+5 glycans
(72N/74S/76N/78S/124N/167N/169T/173S)
(SEQ ID NO: 35)
IAPLQLGNCSVAGWILGNPECNLSINASSWSYIVETPNPENGTCFPGYFADYEELRCQLSSVSSFERF
EIFPKNSSWPNHTVTGVSASCSHNGKSSFYRNLLWLTGKNGLYPNLSKNYTNNKSKEVLVLWGVHHPP
NIGNQRALYHTENAYVSVVSSHYSRRFTPEIAKRPKVRDQEGRINYYWTLLEPGDTIIFEANGNLIAP
WYAFALSRGF
>A/Michigan/45/2015_+4 glycans
(63N/65S/80N/82S/84N/124N)
(SEQ ID NO: 36)
VAPLHLGNCSIAGWILGNPECESLNTSSNWSYIVETSNSDNGTCFPGDFINYEELRCQLSSVSSFERF
EIFPKNSSWPNHDSNKGVTAACPHAGAKSFYKNLIWLVKKGNSYPKLNQSYINDKGKEVLVLWGIHHP
STTADQQSLYQNADAYVFVGTSRYSKKFKPEIATRPKVRDQEGRMNYYWTLVEPGDKITFEATGNLVV
PRYAFTMERNA

HA Antigen Heads with Inter-Bead Stabilizing Mutations

>TH-A/South Carolina/1/1918
(126N/198E/203I/205A/210D/212V/216L)
(SEQ ID NO: 37)
IAPLQLGKCNIAGWLLGNPECDLLLTASSWSYIVETSNSENGTCYPGDFIDYEELRCQLSSVSSFEKF
EIFPKNSSWPNHETTKGVTAACSYAGASSFYRNLLWLTKKGSSYPKLSKSYVNNKGKEVLVLWGVHHP
PTGTDQQSLYQNEDAYVIVASSKYDRVFTPLIAARPKVRDQAGRMNYYWTLLEPGDTITFEATGNLIA
PWYAFALNR
>TH-A/Puerto Rico/8/1934
(58N/76S/84E/198E/203F/205G/210D/212I/216I)
(SEQ ID NO: 38)
IAPLQLGNCSIAGWLLGNPECDPLLSVRSWSYIEETPNSENGICYPGDFIDYEELRCQLSSVSSFERF
EIFPKESSWPNHNTNGVTAACSHEGKSSFYRNLLWLTEKEGSYPKLKNSYVNKKGKEVLVLWGIHHPP
NSKEQQNLYQNENAYVFVGTSNYDRIFTPIIAERPKVRDQAGRMNYYWILLKPGDTIIFEANGNLIAP
MYAFALRRGF
>TH-A/New Caledonia/20/1999 (203L/210D/212V/216I)
(SEQ ID NO: 39)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRVFTPIIAKRPKVRDQEGRINYYWTLLEPGDTIIFEANGNLIAP
WYAFALSRGF
>TH-A/New Caledonia/20/1999_alt1 (203V/210D/212V/216I/218V)
(SEQ ID NO: 42)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYDRVFTPIIVKRPKVRDQEGRINYYWTLLEPGDTIIFEANGNLIAP
WYAFALSRGFG
>TH-A/New Caledonia/20/1999_alt2 (203V/212E/216V/218V)
(SEQ ID NO: 43)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYSREFTPVIVKRPKVRDQEGRINYYWILLEPGDTIIFEANGNLIAP
WYAFALSRGFG
>TH-A/New Caledonia/20/1999_alt3 (203V/210D/2160/218V)
(SEQ ID NO: 44)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYDRRFTPQIVKRPKVRDQEGRINYYWTLLEPGDTIIFEANGNLIAP
WYAFALSRGFG
>TH-A/New Caledonia/20/1999_alt4 (203L/210D/216Q)
(SEQ ID NO: 45)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRRFTPQIAKRPKVRDQEGRINYYWTLLEPGDTIIFEANGNLIAP
WYAFALSRGFG
>TH-A/New Caledonia/20/1999_alt5 (203A/212V/216I/218L)
(SEQ ID NO: 46)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVAVVSSHYSRVFTPIILKRPKVRDQEGRINYYWTLLEPGDTIIFEANGNLIAP
WYAFALSRGFG
>TH-A/New Caledonia/20/1999_alt6 (203A/210D/212V/216T/218L)
(SEQ ID NO: 47)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVAVVSSHYDRVFTPTILKRPKVRDQEGRINYYWTLLEPGDTIIFEANGNLIAP
WYAFALSRGFG
>TH-A/Michigan/45/2015 (101N/198E/200T/205S/210D/212V/216I)
(SEQ ID NO: 48)
VAPLHLGKCNIAGWILGNPECESLSTASSWSYIVETSNSDNGTCFPGNFINYEELRCQLSSVSSFERF
EIFPKTSSWPNHDSNKGVTAACPHAGAKSFYKNLIWLVKKGNSYPKLNQSYINDKGKEVLVLWGIHHP
STTADQQSLYQNEDTYVEVSTSRYDKVEKPIIATRPKVRDQEGRMNYYWTIVEPGDKITFEATGNLVV
PRYAFTMERNA

### Hyperglycosylated H1 Triheads

_>TH-A/South Carolina/1/1918_+5 glycans
(126N/198E/203I/205A/210D/212V/216L)
(SEQ ID NO: 49)
IAPLQLGNCSIAGWLLGNPECDLLLNASSWSYIVETSNSENGTCYPGDFIDYEELRCQLSSVSSFEKF
EIFPKNSSWPNHTTTKGVTAACSYAGASSFYRNLLWLTKKGSSYPKLSKNYTNNKGKEVLVLWGVHHP
PTGTDQQSLYQNEDAYVIVASSKYDRVFTPLIAARPKVRDQAGRMNYYWTLLEPGDTITFEATGNLIA
PWYAFALNR
>TH-A/Puerto Rico/8/1934_+7 glycans
(58N/84E/198E/203F/205G/210D/212I/216I)
(SEQ ID NO: 50)
IAPLQLGNCSIAGWLLGNPECDPLLNVSSWSYIEETPNSENGTCYPGDFIDYEELRCQLSSVSSFERF
EIFPKNSSWPNHTTNGVTAACSHEGKSSFYRNLLWLTEKEGSYPNLTNNYTNKKGKEVLVLWGIHHPP
NSKEQQNLYQNENAYVFVGTSNYDRIFTPIIAERPKVRDQAGRMNYYWTLLKPGDTIIFEANGNLIAP
MYAFALRRGF
>TH-A/New Caledonia/20/1999_+5 glycans
(203L/210D/212V/216I)
(SEQ ID NO: 51)
IAPLQLGNCSVAGWILGNPECNLSINASSWSYIVETPNPENGTCFPGYFADYEELRCQLSSVSSFERF
EIFPKNSSWPNHTVTGVSASCSHNGKSSFYRNLLWLTGKNGLYPNLSKNYTNNKSKEVLVLWGVHHPP
NIGNQRALYHTENAYVLVVSSHYDRVFTPIIAKRPKVRDQEGRINYYWTLLEPGDTIIFEANGNLIAP
WYAFALSR
>TH-A/Michigan/45/2015_+4 glycans
(101N/198E/200T/205S/210D/212V/216I)
(SEQ ID NQ: 52)
VAPLHLGNCSIAGWILGNPECESLNTSSNWSYIVETSNSDNGTCFPGNFINYEELRCQLSSVSSFERF
EIFPKNSSWPNHDSNKGVTAACPHAGAKSFYKNLIWLVKKGNSYPKLNQSYINDKGKEVLVLWGIHHP
STTADQQSLYQNEDTYVFVSTSRYDKVFKPIIATRPKVRDQEGRMNYYWTLVEPGDKITFEATGNLVV
PRYAFTMERNA

### Influenza B Triheads

>TH-B/Brisbane/60/2008
(98R/101L/168E/206K/209Q/213I/257I/259I)
(SEQ ID NO: 53)
EKRGKLCPKCLNCTDLDVALGRPKCTGKIPSARTSELHEERPVTSGCFPIRHDLTKIRCLPNLLRGYE
HIRLSTANVINAENAPGGPYKIGTSGSCPNITNGNGFFATMAWAVPKNDKNKTATEPLTIEVPYICTE
GEDQITVWGFHSDNETQMAKLYGDSKPQQFTSIANGVTTHYVSQIGGFPNQTEDGGLPQSGRIVVDYM
VQKSGKTGIIIYQRGILLPQKVWCASGRSKVIK
>TH-B-Phuket-13
(98R/101L/168E/206K/209Q/213I/257I/259I)
(SEQ ID NO: 54)
RTRGKLCPDCLNCTDLDVALGRPECVGTTPSAKTSELHEREPVTSGCFPIRHDLTKIRCLPNLLRGYE
KIRLSTQNVIDAEKAPGGPYRLGTSGSCPNATSKIGFFATMAWAVPKDNYKNATEPLTVEVPYICTEG
EDQITVWGFHSDNKTQMKSLYGDSKPQQFTSIANGVTTHYVSQIGDEPDQTEDGGLPQSGRIVVDYMM
QKPGKTGIIIYQRGVLLPQKVWCASGRSKVTK

### H3 Trihead

>A/Hong Kong/1/1968
(SEQ ID NO: 55)
KQLDGEDCTLIDALLGDPHCDGFQNETWDQFTERSKAYSNCYPYDVPDYESLRCLVAESGTLEFITEG
FTWTGVTQNGGSNACKRNGESGFFSRLNWLTKSGSTYPVLNVTMPNNDNEDKLYIWGVHHPSTNQEQK
SLYVQESGRVTVSTRRSQQTINPNIGSRPWVRGLSSRISIYYTIVKPGDVLTINSNGNLIAPRGYFKM
KT

### H5 Trihead

>TH-A/Indonesia/5/2005 (203I/210D/212V/214T/216V/221P/244I)
(SEQ ID NO: 56)
VKPLQLRDCSVAGWLLGNPECDEFINVSEWSYIVEKENPTNDLCFPGSENDYEELKCLLSAINHFEKI
QIIPKSSWSDHEASSGVSSACPYLGSPSFFRNVVWLIKKNSTYPTIKKSYNNTNQEDLLVLWGIHHPN
DAAEQTRLYQNPTTYIIIGTSTLDQVLTPVIATRPKVNGQSGRMEFFWTILKPNDAIIFESNGNFIAP
EYAYKIVKKGD

### H2 Trihead

Unverified
>TH-A/Japan/305/1957 (203V/210D/212V/216I/244I)
(SEQ ID NO: 57)
IKPLELGDCSIAGWLLGNPECDRLLSVSEWSYITEKENPRDGLCYPGSFNDYEELKCLLSSVKHFEKV
KILPKDRWTQHTTTGGSRACAVSGNPSFFRNMVWLTEKGSNYPVAKGSYNNTSGEQMLIIWGVHHPND
ETEQRTLYQNVGTYVVVGTSTLDKVSTPIIATRPKVNGQGGRMEFSWTLLDMWDTIIFESTGNLIAPE
YGFKISKR

In some embodiments, the HA protein or immunogenic portion thereof comprises a cysteine residue located at the N-terminus of the heptad motif. In this embodiment, the N-terminus of this motif can be preceded by an “SC” or a “C” sequence which allows for a disulfide bond to be formed with a designed HA head antigen that features a “C” at position 114 relative to SEQ ID NO:1.

In one embodiment, the second domain comprises a polypeptide component of a nanoparticle. Any suitable polypeptide component of a nanoparticle may be used. In various non-limiting embodiments, the polypeptide component may comprise a trimeric polypeptide nanoparticle component as disclosed in WO2021046207 and/or WO2019169120, incorporated by reference herein in their entirety. The second domain may be directly linked to the polypeptide or may be linked via any suitable polypeptide linker, including but not limited to a flexible linker.

In one embodiment, the polypeptide component of the nanoparticle comprises a polypeptide capable for forming a trimer. In an exemplary embodiment, the polypeptide component of a nanoparticle comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:114.

SEQ ID NO: 114
IAELAYLLGELAYKLGEYRIAIRAYRIALKSDPNNAEAWYNLGNAYYKQGRYREAIEYYQKA
LELDPNNAEAWYNLGNAYYERGEYEEAIEYYRKALRLDPNNADAMQNLLNAKMREE
(I53_dn5B)

In one embodiment, the N-terminal I residue of SEQ ID NO: 114 is invariant. The N-terminal I residue helps to rigidly attach the motif to 153 dn5B.

In a further embodiment, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:58-79. In other embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:58 and 60-79. In other embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:58 and 65. The amino acid sequences of these exemplary embodiments are shown in Table 3.

TABLE 3

| SEQ ID | | |
|---|---|---|
| NO | Name | Amino acid sequence |
| 58 | ext-1- | IEHIENEIAELAYLLGELAYKLGEYRIAIRAYRIALKSDENNAEAWYNLGNAY |
| | heptad- | YKQGRYREAIEYYQKALELDPNNAEAWYNLGNAYYERGEYEEAIEYYRKALRL |
| | A_dn5b | DPNNADAMQNLLNAKMREE |
| 59 | ext-2- | IEDKIEEILSKIYHIENEIAELAYLLGELAYKLGEYRIAIRAYRIALKSDPNN |
| | heptad- | AEAWYNLGNAYYKQGRYREAIEYYQKALELDPNNAEAWYNLGNAYYERGEYEE |
| | A_dn5b | AIEYYRKALRLDPNNADAMQNLLNAKMREE |
| 60 | ext-2- | NIENKIEEIEKKIYHIENEIAELAYLLGELAYKLGEYRIAIRAYRIALKSDPN |
| | heptad- | NAEAWYNLGNAYYKQGRYREAIEYYQKALELDPNNAEAWYNLGNAYYERGEYE |
| | B_dn5b | EAIEYYRKALRLDPNNADAMQNLLNAKMREE |
| 61 | ezt-2- | EIEKKIEEIEKKIERIENEIAELAYLLGELAYKLGEYRIAIRAYRIALKSDPN |
| | heptad- | NAEAWYNLGNAYYKQGRYREAIEYYQKALELDPNNAEAWYNLGNAYYERGEYE |
| | C_dn5b | EAIEYYRKALRLDPNNADAMQNLLNAKMREE |
| 62 | ext-2- | NINKKIEEIEKKIYHIENEIAELAYLLGELAYKLGEYRIAIRAYRIALKSDPN |
| | heptad- | NAEAWYNIGNAYYKQGRYREAIEYYQKALELDPNNAEAWYNLGNAYYERGEYE |
| | D_dn5b | EAIEYYRKALRLDPNNADAMQNLLNAKMREE |
| 63 | ext-2- | INKKIEEIEKKIYHIENEIAELAYLLGELAYKLGEYRIAIRAYRIALKSDPNN |
| | heptad- | AEAWYNLGNAYYKQGRYREAIEYYQKALELDPNNAEAWYNLGNAYYERGEYEE |
| | E_dn5b | AIEYYRKALRLDPNNADAMQNLLNAKMREE |
| 64 | ext-2- | EEINKKIEEIEKKIYHIENEIAELAYLLGELAYKLGEYRIAIRAYRIALKSDP |
| | heptad- | NNAEAWYNLGNAYYKQGRYREAIEYYQKALELDPNNAEAWYNLGNAYYERGEY |
| | F_dn5b | EEAIEYYRKALRLDPNNADAMQNLLNAKMREE |
| 65 | ext-2- | IENINSKIYHIENEIAELAYLLGELAYKLGEYRIAIRAYRIALKSDPNNAEAW |
| | heptad- | YNLGNAYYKQGRYREALEYYQKALELDPNNAEAWYNLGNAYYERGEYEEAIEY |
| | G_dn5b | YRKALRLDPNNADAMQNLLNAKMREE |
| 66 | ext-2- | IEAILSKIYHIENEIAELAYLLGELAYKLGEYRIAIRAYRIALKSDPNNAEAW |
| | heptad- | YNLGNAYYKQGRYREAIEYYQKALELDPNNAEAWYNLGNAYYERGEYEEAIEY |
| | H_dn5b | YRKALRLDPNNADAMQNLINAKMREE |
| 67 | ext-2- | IENIASKIYHIENEIAELAYLLGELAYKLGEYRIAIRAYRIALKSDPNNAEAW |
| | heptad- | YNLGNAYYKQGRYREAIEYYQKALELDPNNAEAWYNIGNAYYERGEYEEAIEY |
| | I_dn5b | YRKALRLDENNADAMQNLLNAKMREE |
| 68 | ext-2- | IEQILRKIYHIENEIAELAYLLGELAYKLGEYRIAIRAYRIALKSDENNAEAW |
| | heptad- | YNLGNAYYKQGRYREAIEYYQKALELDPNNAEAWYNLGNAYYERGEYEEAIEY |
| | J_dn5b | YRKALRLDPNNADAMQNLLNAKMREE |
| 69 | ext-3- | IEDKIEEILRKIEEIEKKIYHIENEIAELAYLLGELAYKLGEYRIAIRAYRIA |
| | heptad- | LKSDPNNAEAWYNLGNAYYKQGRYREAIEYYQKALELDPNNAEAWYNLGNAYY |
| | A_dn5b | ERGEYEEAIEYYRKALRLDPNNADAMQNLLNAKMREE |
| 70 | ext-3- | EEIKDKIEEILRKIEEIEKKIYHIENEIAELAYLLGELAYKLGEYRIAIRAYR |
| | heptad- | IALKSDPNNAEAWYNLGNAYYKQGRYREAIEYYQKALELDPNNAEAWYNLGNA |
| | B_dn5b | YYERGEYEEAIEYYRKALRLDPNNADAMQNLLNAKMREE |
| 71 | ext-3- | ILSKIYHIERKIEEILNEIAELAYLLGELAYKLGEYRIAIRAYRIALKSDENN |
| | heptad- | AEAWYNLGNAYYKQGRYREAIEYYQKALELDPNNAEAWYNLGNAYYERGEYEE |
| | C_dn5b | AIEYYRKALRLDPNNADAMQNLLNAKMREE |
| 72 | ext-3- | IEDKIYHIERKIEEILNEIAELAYLLGELAYKLGEYRIAIRAYRIALKSDPNN |
| | heptad- | AEAWYNLGNAYYKQGRYREAIEYYQKALELDPNNAEAWYNLGNAYYERGEYEE |
| | D_dn5b | AIEYYRKALRLDPNNADAMQNLLNAKMREE |
| 73 | ext-3- | NIEDKIYHIERKIEEILNEIAELAYLLGELAYKLGEYRIAIRAYRIALKSDPN |
| | heptad- | NAEAWYNLGNAYYKQGRYREAIEYYQKALELDPNNAEAWYNLGNAYYERGEYE |
| | E_dn5b | EAIEYYRKALRLDPNNADAMQNLLNAKMREE |
| 74 | ext-3- | LSKIYHIERKIEEILNEIAELAYLLGELAYKLGEYRIAIRAYRIALKSDPNNA |
| | heptad- | EAWYNLGNAYYKQGRYREAIEYYQKALELDPNNAEAWYNLGNAYYERGEYEEA |
| | F_dn5b | IEYYRKALRLDPNNADAMQNLLNAKMREE |
| 75 | ext-6- | ILSKIYHIEDKIEEINRKIEKILSKIYHIERKIEEILNEIAELAYLLGELAYK |
| | heptad- | LGEYRIAIRAYRIALKSDPNNAEAWYNLGNAYYKQGRYREAIEYYQKALELDP |
| | A_dn5b | NNAEAWYNIGNAYYERGEYEEAIEYYRKALRLDPNNADAMQNLLNAKMREE |
| 76 | ext-6- | IEDKIYHIEDKIEEINRKIEHILSKIYHIERKIEEILNEIAELAYLLGELAYK |
| | heptad- | LGEYRIAIRAYRIALKSDPNNAEAWYNLGNAYYKQGRYREAIEYYQKALELDP |
| | B_dn5b | NNAEAWYNLGNAYYERGEYEEAIEYYRKALRLDPNNADAMQNLLNAKMREE |
| 77 | ext-6- | NIEDKIYHIEDKIEEINRKIEHILSKIYHIERKIEEILNEIAELAYLLGELAY |
| | heptad- | KLGEYRIAIRAYRIALKSDPNNAEAWYNLGNAYYKQGRYREAIEYYQKALELD |
| | C_dn5b | PNNAEAWYNLGNAYYERGEYEEAIEYYRKALRLDPNNADAMQNLLNAKMREE |
| 78 | ext-6- | IENINSKIYHIEDKIEEINRKIEHILSKIYHIERKIEEILNEIAELAYLLGEL |
| | heptad- | AYKLGEYRIAIRAYRIALKSDPNNAEAWYNIGNAYYKQGRYREATEYYQKALE |
| | D_dn5b | LDPNNAEAWYNLGNAYYERGEYEEAIEYYRKALRLDPNNADAMQNLLNAKMRE |
| | | E |
| 79 | ext-7- | IEDKIEEILSKIYHIEDKIEEINRKIEHILSKIYHIERKIEEILNEIAELAYL |
| | heptad- | LGELAYKLGEYRIAIRAYRIALKSDPNNAEAWYNLGNAYYKQGRYREAIEYYQ |
| | A_dn5b | KALELDPNNAEAWYNLGNAYYERGEYEEAIEYYRKALRLDPNNADAMQNLENA |
| | | KMREE |

In all of these embodiments, the polypeptide may comprise one or more additional domains. In one embodiment where the second domain comprises a component of a nanoparticle, the polypeptide further comprises a third domain comprising a polypeptide antigen. Any suitable polypeptide antigen may be included in this embodiment, including but not limited to an influenza antigen, coronavirus antigen, human immunodeficiency antigens, cytomegalovirus antigens, respiratory syncytial virus antigens, metapneumovirus antigens, parainfluenza virus antigens, Ebola virus antigens, Lassa virus antigens, and Nipah virus antigens, or immunogenic portions thereof. In other embodiments, the antigen may be any antigen as described herein. In one embodiment, the third domain is present N-terminal to the first and second domains.

In a further embodiment, the second domain comprises a polypeptide antigen, and a third domain comprising a polypeptide component of a polypeptide nanoparticle. In this embodiment, the polypeptide antigen and the polypeptide component of a polypeptide nanoparticle may be according to any embodiment or claim described herein. In one such embodiment, the second domain is N-terminal to the first domain, and the third domain is C-terminal to the first domain.

In all of these embodiments, a polypeptide linker may be between any of the domains. In various embodiments, a polypeptide linker is positioned between the second domain and the first domain, and/or between the first domain and the third domain. The linker may be of any length and amino acid composition as suitable for an intended use. In one embodiment, the linker is a GC-rich linker. In other embodiments, the linker may comprise the amino acid sequence selected from the group consisting of SEQ ID NO:80-82.

(SEQ ID NO: 80)
GSGSGSGS
(SEQ ID NO: 81)
GSGSG
(SEQ ID NO: 82)
GSGSGSGECHSP,

In various embodiments, the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:83-113.

### Engineered Stabilizing+Expression Mutations and Disulfide Bonds

### Glycan Knockin Sequon

All residue numbering is based on SEQ ID NO:1 residue numbering:

HA Antigen Heads without Inter-Head Stabilizing Mutations, Fused to dn5b

### H1 Heads

>A/New Caledonia/20/1999_2GCN_dn5b
(SEQ ID NO: 83)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVSVVSSHYSRRFTPEIAKRPKVRDQEGRINYYWILLEPGDTIIFEANGNLIAP
WYAFALSRGFGSGSGKRIENILSKIYHIENEIAELAYLLGELAYKLGEYRIAIRAYRIALKSDPNNAE
AWYNIGNAYYKQGRYREAIEYYQKALELDPNNAEAWYNLGNAYYERGEYEEAIEYYRKALRLDPNNAD
AMQNLLNAKMREE

### H1 Disulfide Only Heads

>A/South Carolina/1/1918_1GCN_dn5b
(SEQ ID NO: 84)
IAPLQLGKCNIAGWLLGNPECDLLLTASSWSYIVETSNSENGTCYPGDFIDYEELRCQLSSVSSFEKF
EIFPKTSSWPNHETTKGVTAACSYAGASSFYRNLLWLTKKGSSYPKLSKSYVNNKGKEVLVLWGVHHP
PTGTDQQSLYQNADAYVSVGSSKYNRRFTPEIAARPKVRDQAGRMNYYWTLLEPGDTITFEATGNLIA
PWYAFALNRGSGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE
>A/Puerto Rico/8/1934_1GCN_dn5b
(SEQ ID NO: 85)
IAPLQLGKCNIAGWLLGNPECDPLLSVRSWSYIVETPNSENGICYPGDFIDYEELRCQLSSVSSFERF
EIFPKESSWPNHNTNGVTAACSHEGKSSFYRNLLWLTEKEGSYPKLKNSYVNKKGKEVLVLWGIHHPP
NSKEQQNLYQNENAYVSVVTSNYNRRFTPEIAERPKVRDQAGRMNYYWTILKPGDTIIFEANGNLIAP
MYAFALRRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>A/New Caledonia/20/1999_1GCN_dn5b
(SEQ ID NO: 86)
IAPLQLGNCSVAGWILGNPECELLISKESWSYIVETPNPENGTCEPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVSVVSSHYSRRFTPEIAKRPKVRDQEGRINYYWILLEPGDTIIFEANGNLIAP
WYAFALSRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>A/Michigan/45/2015_1GCN_dn5b
(SEQ ID NO: 87)
VAPLHLGKCNIAGWILGNPECESLSTASSWSYIVETSNSDNGTCFPGDFINYEELRCQLSSVSSFERF
EIFPKTSSWPNHDSNKGVTAACPHAGAKSFYKNLIWLVKKGNSYPKLNQSYINDKGKEVLVLWGIHHP
STTADQQSLYQNADAYVFVGTSRYSKKFKPEIATRPKVRDQEGRMNYYWTLVEPGDKITFEATGNLVV
PRYAFTMERNAGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE

HA Antigen Heads without Inter-Head Stabilizing Mutations, Fused to dn5b with Hyperglyocsylation

### Hyperglycosylated H1 Disulfide Only Heads

>A/South Carolina/1/1918_+5 glycans_1GCN_dn5b
(63N/65S/81N/125BN/131T/167N/169T)
(SEQ ID NO: 88)
IAPLQLGNCSIAGWLLGNPECDLLLNASSWSYIVETSNSENGTCYPGDFIDYEELRCQLSSVSSFEKF
EIFPKNSSWPNHTTTKGVTAACSYAGASSFYRNLLWLTKKGSSYPKLSKNYTNNKGKEVLVLWGVHHP
PTGTDQQSLYQNADAYVSVGSSKYNRRFTPEIAARPKVRDQAGRMNYYWTLLEPGDTITFEATGNLIA
PWYAFALNRGSGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE
>A/Puerto Rico/8/1934_+7 glycans_1GCN_dn5b
(58N/60S/76N/78S/93T/124N/131T/163N/165T/167N/169T)
(SEQ ID NO: 89)
IAPLQLGNCSIAGWLLGNPECDPLLNVSSWSYIVETPNSENGTCYPGDFIDYEELRCQLSSVSSFERF
EIFPKNSSWPNHTTNGVTAACSHEGKSSFYRNLLWLTEKEGSYPNLTNNYTNKKGKEVLVLWGIHHPP
NSKEQQNLYQNENAYVSVVTSNYNRRFTPEIAERPKVRDQAGRMNYYWTLLKPGDTIIFEANGNLIAP
MYAFALRRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>A/New Caledonia/20/1999_+5 glycans_1GCN_dn5b
(72N/74S/76N/78S/124N/167N/169T/173S)
(SEQ ID NO: 90)
IAPLQLGNCSVAGWILGNPECNLSINASSWSYIVETPNPENGTCFPGYFADYEELRCQLSSVSSFERF
EIFPKNSSWPNHTVTGVSASCSHNGKSSFYRNLLWLTGKNGLYPNLSKNYTNNKSKEVLVLWGVHHPP
NIGNQRALYHTENAYVSVVSSHYSRRFTPEIAKRPKVRDQEGRINYYWTLLEPGDTIIFEANGNLIAP
WYAFALSRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>A/Michigan/45/2015_+4 glycans_1GCN_dn5b (63N/65S/80N/82S/84N/124N)
(SEQ ID NO: 91)
VAPLHLGNCSIAGWILGNPECESLNTSSNWSYIVETSNSDNGTCFPGDFINYEELRCQLSSVSSFERF
EIFPKNSSWPNHDSNKGVTAACPHAGAKSFYKNLIWLVKKGNSYPKLNQSYINDKGKEVLVLWGIHHP
STTADQQSLYQNADAYVFVGTSRYSKKFKPEIATRPKVRDQEGRMNYYWTIVEPGDKITFEATGNLVV
PRYAFTMERNAGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE

HA Antigen Heads with Inter-Head Stabilizing Mutations, Fused to dn5b

### H1 Triheads

>TH-A/South Carolina/1/1918_1GCN_dn5b
(126N/198E/203I/205A/210D/212V/216L)
(SEQ ID NO: 92)
IAPLQLGKCNIAGWLLGNPECDLLLTASSWSYIVETSNSENGTCYPGDFIDYEELRCQLSSVSSFEKF
EIFPKNSSWPNHETTKGVTAACSYAGASSFYRNLLWLTKKGSSYPKLSKSYVNNKGKEVLVLWGVHHP
PTGTDQQSLYQNEDAYVIVASSKYDRVFTPLIAARPKVRDQAGRMNYYWTLLEPGDTITFEATGNLIA
PWYAFALNRGSGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE
>TH-A/Puerto Rico/8/1934_1GCN-dn5b
(58N/76S/84E/198E/203F/205G/210D/2121/216I)
(SEQ ID NO: 93)
IAPLQLGNCSIAGWLLGNPECDPLLSVRSWSYIEETPNSENGICYPGDFIDYEELRCQLSSVSSFERF
EIFPKESSWPNHNTNGVTAACSHEGKSSFYRNLLWLTEKEGSYPKLKNSYVNKKGKEVLVLWGIHHPP
NSKEQQNLYQNENAYVFVGTSNYDRIFTPIIAERPKVRDQAGRMNYYWTLLKPGDTIIFEANGNLIAP
MYAFALRRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>TH-A/New Caledonia/20/1999_1GCN_dn5b (203L/210D/212V/216I)
(SEQ ID NO: 94)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRVFTPIIAKRPKVRDQEGRINYYWTLLEPGDTIIFEANGNLIAP
WYAFALSRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>TH-A/New Caledonia/20/1999_2GCN_dn5b (203L/210D/212V/216I)
(SEQ ID NO: 95)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRVFTPIIAKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMQNLLNAKMREE
>TH-A/New Caledonia/20/1999 6GCN dn5b (203L/210D/212V/216I)
(SEQ ID NO: 96)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRVFTPIIAKRPKVRDQEGRINYYWTLLEPGDTIIFEANGNLIAP
WYAFALSRGFGSGSGSCIENINSKIYHIEDKIEEINRKIEHILSKIYHIERKIEEILNEIAELAYLLG
ELAYKLGEYRIAIRAYRIALKSDPNNAEAWYNLGNAYYKQGRYREAIEYYQKALELDPNNAEAWYNLG
NAYYERGEYEEAIEYYRKALRLDPNNADAMQNLLNAKMREE
>TH-A/Michigan/45/2015_1GCN_dn5b
(101N/198E/200T/205S/210D/212V/216I)
(SEQ ID NO: 97)
VAPLHLGKCNIAGWILGNPECESLSTASSWSYIVETSNSDNGTCFPGNFINYEELRCQLSSVSSFERE
EIFPKTSSWPNHDSNKGVTAACPHAGAKSFYKNLIWLVKKGNSYPKLNQSYINDKGKEVLVLWGIHHP
STTADQQSLYQNEDTYVFVSTSRYDKVEKPIIATRPKVRDQEGRMNYYWTIVEPGDKITFEATGNLVV
PRYAFTMERNAGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE

### Hyperglycosylated H1 Triheads

_>TH-A/South Carolina/1/1918_+5 glycans_1GCN-dn5b
(126N/198E/203I/205A/210D/212V/216L)
(SEQ ID NO: 98)
IAPLQLGNCSIAGWLLGNPECDLLLNASSWSYIVETSNSENGTCYPGDFIDYEELRCQLSSVSSFEKF
EIFPKNSSWPNHTTTKGVTAACSYAGASSFYRNLLWLTKKGSSYPKLSKNYTNNKGKEVLVLWGVHHP
PTGTDQQSLYQNEDAYVIVASSKYDRVFTPLIAARPKVRDQAGRMNYYWTLLEPGDTITFEATGNLIA
PWYAFALNRGSGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE
>TH-A/Puerto Rico/8/1934_+7 glycans_1GCN-dn5b
(58N/84E/198E/203F/205G/210D/212I/216I)
(SEQ ID NO: 99)
IAPLQLGNCSIAGWLLGNPECDPLLNVSSWSYIEETENSENGTCYPGDFIDYEELRCQLSSVSSFERF
EIFPKNSSWPNHTTNGVTAACSHEGKSSFYRNLLWLTEKEGSYPNLTNNYTNKKGKEVLVLWGIHHPP
NSKEQQNLYQNENAYVFVGTSNYDRIFTPIIAERPKVRDQAGRMNYYWILLKPGDTIIFEANGNLIAP
MYAFALRRGFGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>TH-A/New Caledonia/20/1999_+5 glycans_1GCN_dn5b
(203L/210D/212V/216I)
(SEQ ID NO: 100)
IAPLQLGNCSVAGWILGNPECNLSINASSWSYIVETPNPENGTCFPGYFADYEELRCQLSSVSSFERF
EIFPKNSSWPNHTVTGVSASCSHNGKSSFYRNLLWLTGKNGLYPNLSKNYTNNKSKEVLVLWGVHHPP
NIGNQRALYHTENAYVLVVSSHYDRVFTPIIAKRPKVRDQEGRINYYWTLLEPGDTIIFEANGNLIAP
WYAFALSRGEGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE
>TH-A/Michigan/45/2015_+4 glycans_1GCN_dn5b
(101N/198E/200T/205S210D/212V/216I)
(SEQ ID NO: 101)
VAPLHLGNCSIAGWILGNPECESLNTSSNWSYIVETSNSDNGTCFPGNFINYEELRCQLSSVSSFERF
EIFPKNSSWPNHDSNKGVTAACPHAGAKSFYKNLIWLVKKGNSYPKLNQSYINDKGKEVLVLWGIHHP
STTADQQSLYQNEDTYVFVSTSRYDKVFKPIIATRPKVRDQEGRMNYYWTLVEPGDKITFEATGNLVV
PRYAFTMERNAGSGSGSCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE

### Influenza B Triheads

>TH-B/Brisbane/60/2008_1GCN_dn5b
(98R/101L/168E/206K/209Q/213I/257I/259I)
(SEQ ID NO: 102)
EKRGKLCPKCLNCTDLDVALGRPKCTGKIPSARTSELHEERPVTSGCFPIRHDLTKIRCLPNLLRGYE
HIRLSTHNVINAENAPGGPYKIGTSGSCPNITNGNGFFATMAWAVPKNDKNKTATEPLTIEVPYICTE
GEDQITVWGFHSDNETQMAKLYGDSKPQQFTSIANGVTTHYVSQIGGFPNQTEDGGLPQSGRIVVDYM
VQKSGKTGIIIYQRGILLPQKVWCASGRSKVIKGSGSGSGECHSPIEHIENEIAELAYLLGELAYKLG
EYRIAIRAYRIALKSDPNNAEAWYNLGNAYYKQGRYREAIEYYQKALELDPNNAEAWYNLGNAYYERG
EYEEAIEYYRKALRLDPNNADAMQNLLNAKMREE
>TH-B-Phuket-13_1GCN_dn5b (98R/101L/168E/206K/209Q/213I/257I/259I)
(SEQ ID NO: 103)
RTRGKLCPDCLNCTDLDVALGRPECVGTTPSAKTSELHEREPVTSGCFPIRHDLTKIRCLPNLLRGYE
KIRLSTQNVIDAEKAPGGPYRLGTSGSCPNATSKIGFFATMAWAVPKDNYKNATEPLTVEVPYICTEG
EDQITVWGFHSDNKTQMKSLYGDSKPQQFTSIANGVTTHYVSQIGDEPDQTEDGGLPQSGRIVVDYMM
QKPGKTGIIIYQRGVLLPQKVWCASGRSKVTKGSGSGSGECHSPIEHIENEIAELAYLLGELAYKLGE
YRIAIRAYRIALKSDPNNAEAWYNLGNAYYKQGRYREAIEYYQKALELDPNNAEAWYNLGNAYYERGE
YEEAIEYYRKALRLDPNNADAMQNLLNAKMREE

### H3 Trihead

>A/Hong Kong/1/1968_1GCN_dn5b
(SEQ ID NO: 104)
KQLDGEDCTLIDALLGDPHCDGFQNETWDQFTERSKAYSNCYPYDVPDYESLRCLVAESGTLEFITEG
FTWTGVTQNGGSNACKRNGESGFFSRLNWLTKSGSTYPVLNVIMPNNDNEDKLYIWGVHHPSTNQEQK
SLYVQESGRVTVSTRRSQQTINPNIGSRPWVRGLSSRISIYYTIVKPGDVLTINSNGNLIAPRGYFKM
KTGSGSGSCIEHIENEIAELAYLLGELAYKLGEYRIAIRAYRIALKSDPNNAEAWYNLGNAYYKQGRY
REAIEYYQKALELDPNNAEAWYNLGNAYYERGEYEEAIEYYRKALRLDPNNADAMQNLLNAKMREE

### H5 Trihead

>TH-A/Indonesia/5/2005_1GCN_dn5b
(203I/210D/212V/214T/216V/221P/244I)
(SEQ ID NO: 105)
VKPLQLRDCSVAGWLLGNPECDEFINVSEWSYIVEKENPTNDLCFPGSFNDYEELKCLLSAINHFEKI
QIIPKSSWSDHEASSGVSSACPYLGSPSFFRNVVWLIKKNSTYPTIKKSYNNTNQEDLLVLWGIHHPN
DAAEQTRLYQNPTTYIIIGTSTLDQVLTPVIATRPKVNGQSGRMEFFWTILKPNDAIIFESNGNFIAP
EYAYKIVKKGDSGSGSGCIEHIENEIAELAYLLGELAYKLGEYRIAIRAYRIALKSDPNNAEAWYNLG
NAYYKQGRYREAIEYYQKALELDPNNAEAWYNLGNAYYERGEYEEAIEYYRKALRLDPNNADAMQNLL
NAKMREE

### H2 Trihead

Unverified
>TH-A/Japan/305/1957_1GCN_dn5b (203V/210D/212V/216I/244I)
(SEQ ID NO: 106)
IKPLELGDCSIAGWLLGNPECDRLLSVSEWSYITEKENPRDGLCYPGSFNDYEELKCLLSSVKHFEKV
KILPKDRWTQHTTTGGSRACAVSGNPSFERNMVWLTEKGSNYPVAKGSYNNTSGEQMLIIWGVHHPND
ETEQRTLYQNVGTYVVVGTSTLDKVSTPIIATRPKVNGQGGRMEFSWTLLDMWDTIIFESTGNLIAPE
YGFKISKRGSGSGSGSCIEHIENEIAELAYLLGELAYKLGEYRIAIRAYRIALKSDPNNAEAWYNLGN
AYYKQGRYREAIEYYQKALELDPNNAEAWYNLGNAYYERGEYEEAIEYYRKALRLDPNNADAMQNLLN
AKMREE

### “Closed/Flexible”

>TH-A/New Caledonia/20/1999_2GCN_dn5b (203L/210D/212V/216I)
(SEQ ID NO: 107)
IAPLQLGNCSVAGWILGNPECELLISKESWSYIVETPNPENGTCEPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRVFTPIIAKRPKVRDQEGRINYYWTLLEPGDTIIFEANGNLIAP
WYAFALSRGFGSGSGSCIENINSKIYHIENEIARIKKLIGESGGSGGESAELAYLLGELAYKLGEYRI
AIRAYRIALKSDPNNAEAWYNLGNAYYKQGRYREAIEYYQKALELDPNNAEAWYNLGNAYYERGEYEE
AIEYYRKALRLDPNNADAMONLLNAKMREE
>TH-A/New Caledonia/20/1999_alt1_2GCN_dn5b
(203V/210D/212V/216I/218V)
(SEQ ID NO: 108)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYDRVFTPIIVKRPKVRDQEGRINYYWILLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMONLLNAKMREE
>TH-A/New Caledonia/20/1999_alt2_2GCN_dn5b (203V/212E/216V/218V)
(SEQ ID NO: 109)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYSREFTPVIVKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREALEYYQKALELDPNNAEAWYNLGNAYYERGEYEEAIEYYRKALRLDPNNAD
AMQNLLNAKMREE
>TH-A/New Caledonia/20/1999_alt3_2GCN_dn5b (203V/210D/216Q/218V)
(SEQ ID NO: 110)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVVVVSSHYDRRFTPQIVKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMQNLLNAKMREE
>TH-A/New Caledonia/20/1999_alt4_2GCN_dn5b (203L/210D/216Q)
(SEQ ID NO: 111)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVLVVSSHYDRRFTPQIAKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMQNLLNAKMREE
>TH-A/New Caledonia/20/1999_alt5_2GCN_dn5b (203A/212V/2161/218L)
(SEQ ID NO: 112)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVAVVSSHYSRVFTPIILKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMQNLLNAKMREE
>TH-A/New Caledonia/20/1999_alt6_2GCN_dn5b
(203A/210D/212V/216T/218L)
(SEQ ID NO: 113)
IAPLQLGNCSVAGWILGNPECELLISKESWSYIVETPNPENGTCFPGYFADYEELREQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVAVVSSHYDRVFTPTILKRPKVRDQEGRINYYWTLLEPGDTIIFEANGNLIAP
WYAFALSRGFGSGSGSCIENINSKIYHIENEIAELAYLLGELAYKLGEYRIAIRAYRIALKSDPNNAE
AWYNLGNAYYKQGRYREAIEYYQKALELDPNNAEAWYNLGNAYYERGEYEEAIEYYRKALRLDPNNAD
AMQNLLNAKMREE

In another aspect, the disclosure provides mutated HA polypeptides comprising an amino acid sequence at least 75%, 80%, 85%, 90%, 91%, 92%, 93%, 94%, 95%, 96%, 97%, 98%, or 99% identical to the amino acid sequence of SEQ ID NO:1, wherein the HA protein or immunogenic portion thereof includes 1, 2, 3, 4, or all 5 of the following amino acid residues relative to SEQ ID NO: 1 residues 51-264 (the head domain, bolded in SEQ ID NO: 1) when aligned by protocol 1 or protocol 2: 107C, 203L, 210D, 212V or I (or 212V), and/or 216I.

These mutations are shown in the examples to increase trimer formation.

In one embodiment, the HA protein or immunogenic portion thereof further includes 1, 2, 3, 4, 5, 6, 7, 8, 9, 10, 11, or all 12 of the following amino acid residues, residue numbering relative to SEQ ID NO: 1: 58N, 101N, 126N, 203I/F/L/V/A, 205A or 205G, 212E, 214T, 216L/V/Q/T, 218V or 218L, 221P, and/or 244I. Each of these mutations is shown in the examples to stabilize trimer formation.

In a further embodiment, the HA mutated protein or immunogenic portion thereof includes a combination of amino acid residues selected from the group consisting of, residue numbering relative to SEQ ID NO:1:
126N/203I/205A/210D/212V or I (or 212V)/216L,
58N/203F/205G/210D/212V or I (or 212V)/216I,
203L/210D/212V or I (or 212V)/216I,
203V/210D/212V or I (or 212V)/216I/218V,
203V/212E/216V/218V,
203V/210D/216Q/218V,
203L/210D/216Q,
203A/212V or I (or 212V)/216I/218L,
203A/210D/212V or I (or 212V)/216T/218L,
101N/210D/212V or I (or 212V)/216I,
203I/210D/212V or I (or 212V)/214T/216V/221P/244I, and
203V/210D/212V or I (or 212V)/216I/244I.

In another embodiment, the mutated HA protein or immunogenic portion thereof further includes 1, 2, 3, 4, 5, 6, 7, 8, 9, 10, 11, 12, or all 13 of the following amino acid residues, residue numbering relative to SEQ ID NO:1: 62E, 70E, 76S, 78G, 78S, 84E, 142N, 143G, 144E, 192K, 198E, 200T, and/or 205S. Each of these mutations is shown in the examples to enhance antigen expression.

In a further embodiment, the mutated HA protein or immunogenic portion thereof includes a combination of amino acid residues selected from the group consisting of the following, residue numbering relative to SEQ ID NO:1:
198E,
76S/84E/198E,
198E/200T/205S,
62E/78G/142N/143G/144E/192K, and/or
70E/78S.

Each of these combinations of mutations is shown in the examples to enhance antigen expression.

In one embodiment, the mutated HA protein or immunogenic portion thereof further includes a combination of amino acid residues selected from the group consisting of the following, residue numbering relative to SEQ ID NO: 1:
63N/65S/81N/125BN/131T/167N/169T,
58N/60S/76N/78S/93T/124N/131T/163N/165T/167N/169T,
72N/74S/76N/78S/124N/167N/169T/173S,
63N/65S/80N/82S/84N/124N, or
63N/65S/81N/125BN/131T/167N/169T.

These mutations add glycosylation sites to the antigens.

In another embodiment, the mutated HA protein or immunogenic portion thereof comprises Y or F at residue 95, residue numbering relative to SEQ ID NO:1. In a further embodiment, the HA protein or immunogenic portion thereof comprises amino acid residues selected from those listed in Table 2 at the listed positions, residue numbering relative to SEQ ID NO: 1.

In one embodiment of these various embodiments, the mutant HA polypeptides comprise an amino acid sequence at least 75%, 80%, 85%, 90%, 91%, 92%, 93%, 94%, 95%, 96%, 97%, 98%, or 99% identical to the amino acid sequence of SEQ ID NO: 1 or SEQ ID NO: 5, wherein the N-terminal methionine residue is optional and may be present or absent. In another embodiment, the mutant HA polypeptides comprise an amino acid sequence at least 75%, 80%, 85%, 90%, 91%, 92%, 93%, 94%, 95%, 96%, 97%, 98%, or 99% identical to the amino acid sequence of SEQ ID NO: 1, wherein the N-terminal ATYA is optional and may be present or absent. In a further embodiment, the mutant polypeptides comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 33-39 and 42-52, wherein at least 1, 2, 3, 4, 5, 6, 7, 8, 9, 10, 11, 12, 13, 14, or 15 of the bold-faced residue(s) is/are present in the polypeptide.

>A/Puerto Rico/8/1934_+7 glycans_1GCN_dn5b
(58N/60S/76N/78S/93T/124N/131T/163N/165T/167N/169T)
IAPLQLGNCSIAGWLLGNPECDPLLNVSSWSYIVETPNSENGTCYPGDFIDYEELRCQLSSVSSFERFEIFPKNS
SWPNHTTNGVTAACSHEGKSSFYRNLLWLTEKEGSYPNLTNNYTNKKGKEVLVLWGIHHPPNSKEQQNLYQNENA
YVSVVTSNYNRRFTPEIAERPKVRDQAGRMNYYWTLLKPGDTIIFEANGNLIAPMYAFALRRGF

In another aspect, the disclosure provides mutated HA polypeptides, comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:29-32 and 53-57, wherein mutations relative to wild type are noted in bold and are listed above the sequence, and wherein the at least 1, 2, 3, 4, 5, 6, 7, or all of the mutations are present in the polypeptide.

### H1 Disulfide Only Heads

>A/South Carolina/1/1918
(SEQ ID NO: 29)
IAPLQLGKCNIAGWLLGNPECDLLLTASSWSYIVETSNSENGTCYPGDFIDYEELRCQLSSVSSFEKF
EIFPKTSSWPNHETTKGVTAACSYAGASSFYRNLLWLTKKGSSYPKLSKSYVNNKGKEVLVLWGVHHP
PTGTDQQSLYQNADAYVSVGSSKYNRRFTPEIAARPKVRDQAGRMNYYWTLLEPGDTITFEATGNLIA
PWYAFALNR
>A/Puerto Rico/8/1934
(SEQ ID NO: 30)
IAPLQLGKCNIAGWLLGNPECDPLLSVRSWSYIVETPNSENGICYPGDFIDYEELRCQLSSVSSFERF
EIFPKESSWPNHNINGVTAACSHEGKSSFYRNLLWLTEKEGSYPKLKNSYVNKKGKEVLVLWGIHHPP
NSKEQQNLYQNENAYVSVVTSNYNRRFTPEIAERPKVRDQAGRMNYYWTLLKPGDTIIFEANGNLIAP
MYAFALRRGF
>A/New Caledonia/20/1999
(SEQ ID NO: 31)
IAPLQLGNCSVAGWILGNPECELLISKESWSYIVETPNPENGTCFPGYFADYEELRCQLSSVSSFERF
EIFPKESSWPNHTVTGVSASCSHNGKSSFYRNLLWLTGKNGLYPNLSKSYVNNKEKEVLVLWGVHHPP
NIGNQRALYHTENAYVSVVSSHYSRRFTPEIAKRPKVRDQEGRINYYWTLLEPGDTIIFEANGNLIAP
WYAFALSRGF
>A/Michigan/45/2015
(SEQ ID NO: 32)
VAPLHLGKCNIAGWILGNPECESLSTASSWSYIVETSNSDNGTCFPGDFINYEELRCQLSSVSSFERF
EIFPKTSSWPNHDSNKGVTAACPHAGAKSFYKNLIWLVKKGNSYPKINQSYINDKGKEVLVLWGIHHP
STTADQQSLYQNADAYVFVGTSRYSKKFKPEIATRPKVRDQEGRMNYYWTLVEPGDKITFEATGNLVV
PRYAFTMERNA

### Influenza B Triheads

>TH-B/Brisbane/60/2008
(98R/101L/168E/206K/209Q/213I/257I/259I)
(SEQ ID NO: 53)
EKRGKLCPKCLNCTDLDVALGRPKCTGKIPSARTSELHEERPVTSGCFPIRHDLTKIRCLPNLLRGYE
HIRLSTHNVINAENAPGGPYKIGTSGSCPNITNGNGFFATMAWAVPKNDKNKTATEPLTIEVPYICTE
GEDQITVWGFHSDNETQMAKLYGDSKPQQFTSIANGVTTHYVSQIGGFPNQTEDGGLPQSGRIVVDYM
VQKSGKTGIIIYQRGILLPQKVWCASGRSKVIK
>TH-B-Phuket-13
(98R/101I/168E/206K/209Q/213I/257I/259I)
(SEQ ID NO: 54)
RTRGKLCPDCLNCTDLDVALGRPECVGTTPSAKTSELHEREPVTSGCFPIRHDLTKIRCLPNLLRGYE
KIRLSTQNVIDAEKAPGGPYRLGTSGSCPNATSKIGFFATMAWAVPKDNYKNATEPLTVEVPYICTEG
EDQITVWGFHSDNKTQMKSLYGDSKPQQFTSIANGVTTHYVSQIGDFPDQTEDGGLPQSGRIVVDYMM
QKPGKTGIIIYQRGVLLPQKVWCASGRSKVTK

### H3 Trihead

>A/Hong Kong/1/1968
(SEQ ID NO: 55)
KQLDGEDCTLIDALLGDPHCDGFQNETWDQFTERSKAYSNCYPYDVPDYESLRCLVAESGTLEFITEG
FTWTGVTQNGGSNACKRNGESGFFSRLNWLTKSGSTYPVLNVTMPNNDNEDKLYIWGVHHPSTNQEQK
SLYVQESGRVTVSTRRSQQTINPNIGSRPWVRGLSSRISIYYTIVKPGDVLTINSNGNLIAPRGYFKM
KT

### H5 Trihead

>TH-A/Indonesia/5/2005
(2031/210D/212V/214T/216V/221P/244I)
(SEQ ID NO: 56)
VKPLQLRDCSVAGWLLGNPECDEFINVSEWSYIVEKENPTNDLCFPGSFNDYEELKCLLSAINHFEKI
QIIPKSSWSDHEASSGVSSACPYLGSPSFFRNVVWLIKKNSTYPTIKKSYNNTNQEDLLVLWGIHHPN
DAAEQTRLYQNPTTYIIIGTSTLDQVLTPVIATRPKVNGQSGRMEFFWTILKPNDAIIFESNGNFIAP
EYAYKIVKKGD

### H2 Trihead

Unverified
>TH-A/Japan/305/1957 (203V/210D/212V/216I/244I)
(SEQ ID NO: 57)
IKPLELGDCSIAGWLLGNPECDRLLSVSEWSYITEKENPRDGLCYPGSENDYEELKCLLSSVKHFEKV
KILPKDRWTQHTTTGGSRACAVSGNPSFERNMVWLTEKGSNYPVAKGSYNNTSGEQMLIIWGVHHPND
ETEQRTLYQNVGTYVVVGTSTLDKVSTPIIATRPKVNGQGGRMEFSWTLLDMWDTIIFESTGNLIAPE
YGFKISKR

In another aspect, the disclosure provides nucleic acids encoding a polypeptide of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In one embodiment of the nucleic acids of the disclosure, the nucleic acid comprises mRNA. This embodiment is particularly useful as a vaccine. After mRNA administration (such as by injection) and uptake by antigen-presenting cells (APCs), the polypeptide is expressed in APCs and displayed for the immune response. Various modifications of mRNA may be used in order to counter the degradation of a mRNA therapeutic or vaccine disclosed herein. In one embodiment, the RNA comprises nucleoside-modified RNA. In other embodiments, the nucleic acid (DNA or RNA) comprises a poly A tail (DNA). In a further embodiment, the nucleic acid may comprise 5′ and/or 3″ untranslated regions.

In another aspect, disclosure provides expression vectors comprising the isolated nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In a further aspect, the present disclosure provides cells comprising the polypeptide, the nanoparticle, the composition, the nucleic acid, and/or the expression vector of any embodiment or combination of embodiments of the disclosure, wherein the cells can be either prokaryotic or eukaryotic, such as mammalian cells. In one embodiment the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In one aspect, the disclosure provides nanoparticles comprising a plurality of the polypeptides of any embodiment of the disclosure that include a nanoparticle component. In some embodiments, the nanoparticles comprise the polypeptide of any embodiment of the disclosure that includes a third domain such that the polypeptide comprises both a polypeptide antigen and a polypeptide component of a nanoparticle.

The plurality of polypeptides may all be identical, or may contact different polypeptides. In some embodiments, the different polypeptides comprises a unique immunogenic portion from a different Type, Group, subtype, or strain of influenza virus. In a further embodiment, the nanoparticle displays an immunogenic portion of 3, 4, or more different HA proteins. In one embodiment, the nanoparticle displays an immunogenic portion of 2 or more different immunogenic regions of HA proteins selected from the group consisting of ectodomain, stem, stabilized stem, and head regions. In some embodiments, the immunogenic portion of the different HA proteins are selected from the group consisting of immunogenic portions of influenza A and influenza B HA proteins. In other embodiments, the immunogenic portion of the different HA proteins are selected from the group consisting of immunogenic portions of an H1, H2, H3, H4, H5, H6, H7, H8, H9, H10, H11, H12, H13, H14, H15, H16 H17, and H18 HA protein. In further embodiments, the immunogenic portion of the different HA proteins comprise immunogenic portions of HA proteins from strains including but not limited to H10N4, H10N5, H10N7, H10N8, H10N9, H11N1, H11N3, H11N2, H11N4, H11N6, H11N8, H11N9, H12N1, H12N4, H12N5, H12N8, H13N2, H13N3, H13N6, H13N7, H14N5, H14N6, H15N8, H15N9, H16N3, H1N1, H1N2, H1N3, H1N6, H1N9, H2N1, H2N2, H2N3, H2N5, H2N7, H2N8, H2N9, H3N1, H3N2, H3N3, H3N4, H3N5, H3N6, H3N8, H3N9, H4N1, H4N2, H4N3, H4N4, H4N5, H4N6, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N4, H8N5, H9N1, H9N2, H9N3, H9N5, H9N6, H9N7, H9N8, and H9N9. In various further embodiments, the immunogenic portion of the HA proteins comprise an immunogenic portion of each of (a) one influenza A Group 1 HA, (b) one influenza A Group 2 HA, and (c) two influenza B HAs; or (a) one H1 HA, (b) one H3 HA, and (c) one or two influenza B Has; or (a) A/Michigan/45/2015 (H1N1), (b) A/Hong Hong/480I/2014 (H3N2), (c) B/Brisbane/60/2008 (Victoria lineage), and (d) B/Phuket/3073/2013 (Yamagata lineage); or (a) A/Idaho/07/2018 (H1N1) pdm09-like virus, (b) A/Perth/1008/2019 (H3N2)-like virus (updated), (c) B/Colorado/06/2017-like (Victoria lineage) virus (updated), and optionally including (d) B/Phuket/3073/2013-like (Yamagata lineage) virus; or (a) the composition of seasonal influenza virus vaccine of 2020 (Southern hemisphere), (b) A/Brisbane/02/2018 (H1N1) pdm09-like, (c) A/South Australia/34/2019 (H3N2)-like, (d) B/Washington/02/2019-like (Victoria lineage), and (e) B/Phuket/3073/2013-like (Yamagata lineage).

In another embodiment, the different polypeptide antigens include HA polypeptides, or immunogenic fragments thereof, that differ at 1, 2, 3, 4, 5, 6, 7, 8, 9, 10, 11, or all 12 of the positions (residue numbering relative to SEQ ID NO: 1) listed in Table 2, optionally wherein the positions include an amino acid residue noted at the specific position in Table 2.

In some embodiments, the nanoparticle comprises:
(a) a plurality of first assemblies, each first assembly comprising a plurality of identical first polypeptides, wherein the first polypeptides comprise an amino acid sequence having at least 50%, 55%, 60%, 65%, 70%, 75%, 80%, 85%, 90%, 95%, 96%, 97%, 98%, 99%, or 100% sequence identity to the amino acid sequence selected from the group consisting of SEQ ID NOS: 2-4, where residues in parentheses are optional and may be present or absent;

>I53_dn5A*
(SEQ ID NO: 2)
(MG)KYDGSKLRIGILHARWNAEIILALVLGALKRLQEFGVKRENIIIETVPGSFEL
PYGSKLEVEKQKRLGKPLDAIIPIGVLIKGSTMHFEYICDSTTHQLMKLNFELGIPV
IFGVLTCLTDEQAEARAGLIEGKMHNHGEDWGAAAVEMATKFN;
>I53_dn5A.1
(SEQ ID NO: 3)
(MG)KYDGSKLRIGILHARGNAEIILALVLGALKRLQEFGVKRENIIIETVPGSFEL
PYGSKLFVEKQKRLGKPLDAIIPIGVLIRGSTPHEDYIADSTTHOLMKLNFELGIPV
IFGVITADTDEQAEARAGLIEGKMHNHGEDWGAAAVEMATKFN;
and
>I53_dn5A.2
(SEQ ID NO: 4)
(MG)KYDGSKLRIGILHARGNAEIILELVLGALKRLQEFGVKRENIIIETVPGSFEL
PYGSKLFVEKQKRLGKPLDAIIPIGVLIRGSTAHFDYIADSTTHQLMKLNFELGIPV
IFGVLTTESDEQAEERAGTKAGNHGEDWGAAAVEMATKFN;

and
(b) a plurality of second assemblies, each second assembly comprising a plurality of second polypeptides, wherein the second polypeptides comprise the polypeptide of any embodiment of the disclosure that includes a third domain such that the polypeptide comprises both a polypeptide antigen and a polypeptide component of a nanoparticle, wherein the polypeptide component of a nanoparticle comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO: 114 (I53_dn5B);
wherein the plurality of first assemblies non-covalently interact with the plurality of second assemblies to form a nanostructure; and
wherein the nanostructure displays multiple copies of one or more immunogenic polypeptide antigens, on an exterior of the nanostructure.

In this embodiment, the polypeptide component of the nanoparticle (I53_dn5B) is one that non-covalently interacts with the recited polypeptides of the first assembly (I53_dn5A). In this embodiment, the polypeptide of the disclosure comprises the heptad motif linked (directly or via a linker) to one or both of the polypeptide antigen or the polypeptide component of the nanoparticle (i.e.: Antigen-heptad-NP component, or NP component-heptad-antigen). In one embodiment, the second polypeptides comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 58-79, or SEQ ID NO:58 and 60-19, or SEQ ID NO: 58 and 65. In this embodiment, the polypeptide antigen may be any antigen, including but not limited to those disclosed herein. In another embodiment, the second polypeptides comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 83-113, which include various HA antigens. In all of these embodiments, the second polypeptides in the plurality of second polypeptides may be the same or may include different second polypeptides. For example, in some embodiments, the polypeptide component of each of the second polypeptides may be identical, while the polypeptide antigen may be the same or may include different polypeptide antigens. In a further embodiment, the heptad repeat of each second polypeptide may be the same, or may differ.

In another aspect, the disclosure provides pharmaceutical compositions comprising
(a) the polypeptide, nucleic acid, recombinant expression vector, cell, and/or nanoparticle of any embodiment or combination of embodiments herein; and
(b) a pharmaceutically acceptable carrier.

As shown in the examples that follow, the compositions provide, for example, improved vaccines. The polypeptides disclosed permit the most rigid attachment of an antigen to a protein nanoparticle to date. The compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptide antigen may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCATI, pCMVN, Peptomer-NP, Plcuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In one embodiment, the pharmaceutical composition comprises:
(a) the mRNA of any embodiment herein; and
(b) a cationic lipid such as a liposome, or a cationic protein such as protamine.

In another embodiment, the pharmaceutical composition comprises:
(a) the polypeptide of any embodiment herein; and
(b) a pharmaceutically acceptable carrier.

In another embodiment, the pharmaceutical composition comprises:
(a) a plurality of the nanoparticle of embodiment herein; and
(b) a pharmaceutically acceptable carrier.

The pharmaceutical compositions can be used, for example, as vaccines. Thus, in another embodiment, the disclosure provides vaccines comprising the polypeptide, nucleic acid, recombinant expression vector, cell, nanoparticle, or composition of any embodiment herein.

In another aspect, the disclosure provides methods to vaccinate a subject against an infectious agent, including but not limited to the influenza virus, the method comprising administering to the subject an effective amount of the polypeptide, nucleic acid, recombinant expression vector, cell, nanoparticle, or composition of any embodiment herein to limit development of an infection. In some embodiments, the administering elicits an immune response in the subject, such that the subject is protected against infection by an infectious agent, including but not limited to a heterologous influenza virus. In some embodiments, the methods limit development of an infection, including but not limited to an influenza infection. As used herein, “limiting development” includes, but is not limited to accomplishing one or more of the following: (a) generating an immune response (antibody and/or cell-based) to the infectious agent in the subject; (b) generating neutralizing antibodies against the infectious agent in the subject (b) limiting build-up of infectious agent titer in the subject after exposure to the infectious agent; and/or (c) limiting or preventing development of infectious agent symptoms after infection.

As used herein, an “effective amount” refers to an amount of the immunogenic composition that is effective for treating and/or limiting infection. The polypeptide, nanoparticle, composition, nucleic acid, pharmaceutical composition, or vaccine of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

The subject may be any subject at risk of infection. In one embodiment, the subject is a mammalian subject. In another embodiment, the subject is a human subject.

In another aspect, the disclosure provides methods of detecting anti-infectious agent antibodies, including but not limited to anti-influenza virus antibodies, comprising:
a. contacting at least a portion of a sample being tested for the presence of anti-infectious agent antibodies, such as influenza antibodies, with a polypeptide or nanoparticle of any embodiment herein; and
b. detecting the presence of an antibody-nanoparticle complex;
wherein the presence of an antibody-nanoparticle complex indicates that the sample contains anti-infectious agent antibodies, such as anti-influenza antibodies.

In a further aspect, the disclosure provides methods to identify a subject anti-infectious agent antibodies, including but not limited to anti-influenza virus antibodies, comprising:
(a) contacting a sample from a subject being tested with a polypeptide or nanoparticle of any embodiment herein; and,
(b) analyzing the contacted sample for the presence of an antibody-nanoparticle complex;
wherein the presence of an antibody-nanoparticle complex indicates the subject has anti-infectious agent antibodies, such as anti-influenza antibodies.

In another embodiment, the disclosure provides methods to identify a subject that has been exposed to an infectious agent, including but not limited to an influenza virus, the method comprising:
(a) contacting at least a portion of a sample from a subject being tested with a polypeptide or nanoparticle of any embodiment herein; and,
(b) analyzing the contacted sample for the presence or level of an antibody/nanoparticle complex, wherein the presence or level of antibody-nanoparticle complex indicates the presence or level of recent anti-infectious agent antibodies, such as anti-influenza antibodies; and
(c) comparing the recent antibody level with a past antibody level;
wherein an increase in the recent antibody level over the past antibody level indicates the subject has been exposed to infectious agent, such as influenza virus subsequent to determination of the past antibody level.

In one embodiment, the disclosure provides methods for measuring the response of a subject to a vaccine, the method comprising:
(a) administering to the subject a vaccine for an infectious agent, including but not limited to influenza virus;
(b) contacting at least a portion of a sample from the subject with the polypeptide or nanoparticle of any preceding claim;
(c) analyzing the contacted sample for the presence or level of an antibody/nanoparticle complex, wherein the presence or level of antibody-nanoparticle complex indicates the presence or level of recent anti-infectious agent antibodies;
wherein an increase in the level of antibody in the sample over the pre-vaccination level of antibody in the subject indicates the vaccine induced an immune response in the subject.

In one embodiment of all of these embodiments, the sample may a body fluid, including but not limited to blood, plasma, serum, lacrimal fluid and saliva.

### Examples. Design of a Modular Nanoparticle Platform for Tunable Antibody Responses Against the Influenza Hemagglutinin Head Domain

### Summary

Influenza vaccines are characteristic in their elicitation of narrowly-specific responses against the head domain of the hemagglutinin glycoprotein (HA). Here we use structure-based computational protein design strategies to design a nanoparticle-based platform that allows for multiple levels of control over epitopes prioritized in responses against the HA bead domain. HA head domains can either be presented as monomers or in their native-like closed trimeric formation that structurally occludes access to the interface region between heads. Further, the designed platform features a rigidly-bridging extension domain that can modularly be shrunk or extended to precisely control spacing between antigens. Smaller spacings between adjacent head antigens on the nanoparticle surface was found to correlate with improved neutralization potency as well as binding breadth across diverse H1N1 HAs.

### INTRODUCTION

An improved understanding of how to influence antibody responses against desired epitopes on any single arbitrary HA head without modifications to its intrinsic antigenicity could provide vital information for the development of improved head-targeting influenza vaccines.

Studying potential structural correlates of immunogenicity and leveraging such spatial properties for immunogen design has been difficult due to inherent structural flexibility in many particulate or oligomeric systems.

Here, we design a model system for bespoke presentation of the HA head domain on the surface of protein nanoparticles that allows for control over both the local oligomeric state of head domains as well as their precise spatial organization. Alterations to both features allows the immunogenicity of different epitopes on the HA head to be modulated. In addition, we designed glycosylation sites and hypervariable sequences onto several strains of HA in order to more precisely direct the resulting immune response. This work provides novel approaches to vaccine development for influenza, and other pathogens, through providing methods and design principles for customized particulate immunogen design.

### Results

We aimed to design a platform that could allow for customizable targeting of different epitopes on the HA head domain of an arbitrary individual strain while minimally altering key antigenic sites on the head itself. HA is split into two genetic units, with HA1 largely containing the head domain and HA2 presenting the majority of the stem domain. HA natively forms as a trimer in which the HA2 region contains the majority of interprotomeric contacts, while the head domains maintain relatively smaller numbers of contacts with other heads and can experience separated “breathing” (Benton et al. 2020; Das et al. 2018). When expressed on their own without the stem domain, HA head domains are subsequently monomeric. Even when attached to other multimeric structures, such as protein nanoparticles, HA head domains do not natively form reliable contacts and leave inter-head interface regions largely exposed (Kanekiyo et al. 2019). Given that broadly conserved epitopes present at the HA head interface could either be considered desirable targets or limitations to the focus on more valuable epitopes that can host neutralizing responses, we envisioned a modular way of controlling whether the HA head was monomeric or held in the closed trimeric state observed in common ectodomain structures, all in the absence of HA2. Two possible strategies were considered: design of interactions between HA head domains and a different rigid trimeric base which imitates HA2, and design of interactions within head domains to favor the trimeric state.

To further gain control over epitopes targeted on head domain, we hypothesized that precise control of the geometry in which head domains are displayed on a nanoparticle surface could be used to preferentially highlight different epitopes for humoral focus. Given that spatial features of multivalent antigens arrays, whether on pathogens or other designed surfaces, have been suggested to influence humoral responses, we outlined a strategy for displaying the HA head in distinct rigid spacings. Symmetrical helical bundles can contain structurally repeating motifs, such as heptads which repeat structurally identical backbones every seven amino acids. This modularity can be exploited by protein design to concatenate heptad repeats into rigid units with arbitrary lengths. A GCN4 bundle was used as a starting point for designing “heptad motifs” in this work (SEQ ID NO:7). A redesigned C3 homotrimeric GCN4 helical bundle of SEQ ID NO: 13 features such heptad repeats, and is an example of the “heptad motifs” disclosed herein. We used this helical bundle to mediate a connection between HA heads and a protein nanoparticle surface that could support the heads in their native closed trimeric state, while reengineering the length of the bundle to alter spacing between the HA heads and the nanoparticle surface.

A preliminary trimeric head (“TriHead”) design (SEQ ID NO:95) was developed using the H1N1 strain A/New Caledonia/20/1999 (NC99), which sequentially features the receptor binding domain (RBD) of the HA head (residues 51-264 containing the Y95F mutation [Y98F in H3 numbering]), the ext-2-heptad motif of SEQ ID NO:13, and the trimeric component of the I53_dn5 nanoparticle (I53_dn5B) (SEQ ID NO: 107). The RBD was connected to the heptad motif using a flexible six-residue linker. The heptad motif was designed to match residues that normally contact the HA head with the intention that the RBDs may naturally recognize this interface to drive trimerization. The I53_dn5 nanoparticle was selected due to the N terminus of I53_dn5B containing helical regions that closely match the C-terminal end of GCN4, and the hydrophobic core of both helical structures containing a similar pattern of hydrophobic residues. Rather than connecting the two domains using a flexible genetic linker, the C-terminal end of GCN4 was merged with the N-terminal helices of the I53_dn5 trimer by replacing terminal hydrophobic residues GCN4 with complementary positions in the I53_dn5B N-terminal helix to generate the heptad motif, and blend them into one continuous helical bundle. This design of SEQ ID NO:95 was successfully secreted from HEK293F cells, purified by immobilized metal affinity chromatography and confirmed as a trimer by size exclusion chromatography (SEC) (not shown). To infer whether RBDs in this fusion protein formed the closed trimeric state, binding to the non-neutralizing interface-directed antibody D2 H1-1/H3-1 (J. Lee et al. 2016) was measured using biolayer interferometry (BLI), which demonstrated that the interface region was solvent-exposed and accessible for antibody recognition (FIG. 1B).

We next asked whether strengthened interactions with the heptad motif could favor the trimeric closed state of the RBDs. Computational modeling with ROSETTA™ was used to identify a favorable disulfide bond between the RBD at position 107 and the N-terminal portion of the heptad motif. This construct of SEQ ID NO:86 was expressed and purified and similarly identified as a trimer, however binding to D2 H1-1/H3-1 was still detected (data not shown). Greater addition of designed interactions between the RBDs was therefore investigated for maintaining trimer closure. ROSETTA™ was used to build hydrophobic interfaces between the RBDs, while not directly mutating epitopes contacted by D2 H1-1/H3-1 or other interface-directed antibodies. Multiple designs were successfully expressed at small-scale and, in contrast to previous designs, showed elimination of binding to D2 H1-1/H3-1 while maintaining binding to the mAb C05 in supernatant BLI measurements, in all suggesting improved closure of the head antigens in a trimeric while maintaining antigenicity of the RBS (SEQ ID NOs 95, 108-113) (data not shown). One candidate with such BLI metrics of SEQ ID NO 95 featuring 203L/210D/212V/216I mutations at the trimerization interface (FIG. 1A) was selected for scale-up and purification by SEC for more detailed analysis. The combination of binding to C05 and elimination of binding to D2 H1-1/H3-1 was confirmed with BLI using the purified trimeric component of this design (FIG. 1B).

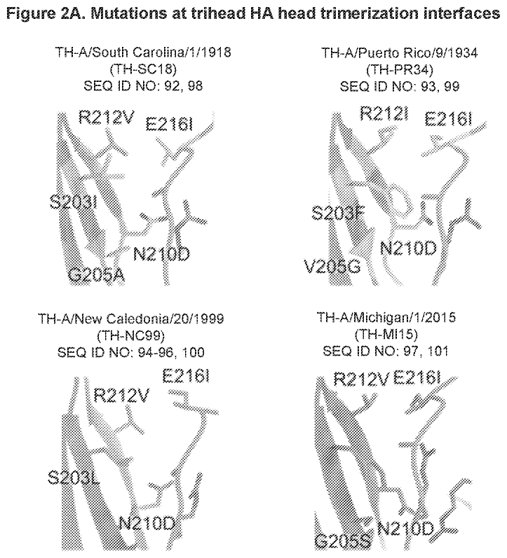

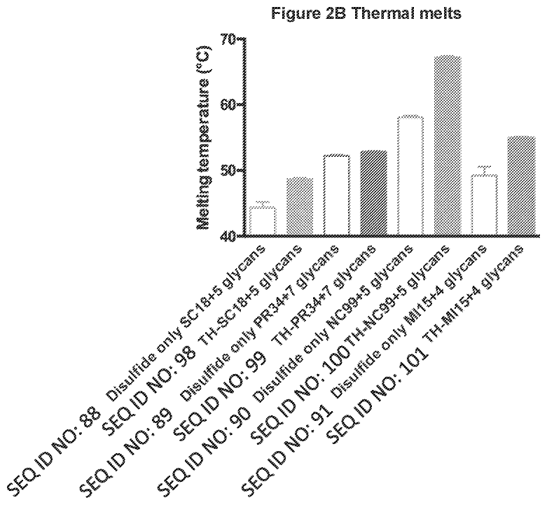

Purified protein for this design (SEQ ID NO: 95) was mixed with the complementary I53_dn5A. 1 pentameric component to drive nanoparticle assembly, and purified nanoparticles were analyzed by cryo-electron microscopy (EM) (FIG. 2A), EM 2D class averages showed density consistent with closed trimeric RBDs rigidly attached to the nanoparticle surface (FIG. 1C), further confirming success of the design. 3D reconstruction resolved density at 8.1 Å, with the size of the density confirmed to match closed trimeric RBDs (FIG. 7A). While clear secondary structure could be resolved for the nanoparticle interior, there was a notable drop in local resolution for the RBDs, suggesting that a small amount of flexibility is present between the nanoparticle and the antigens.

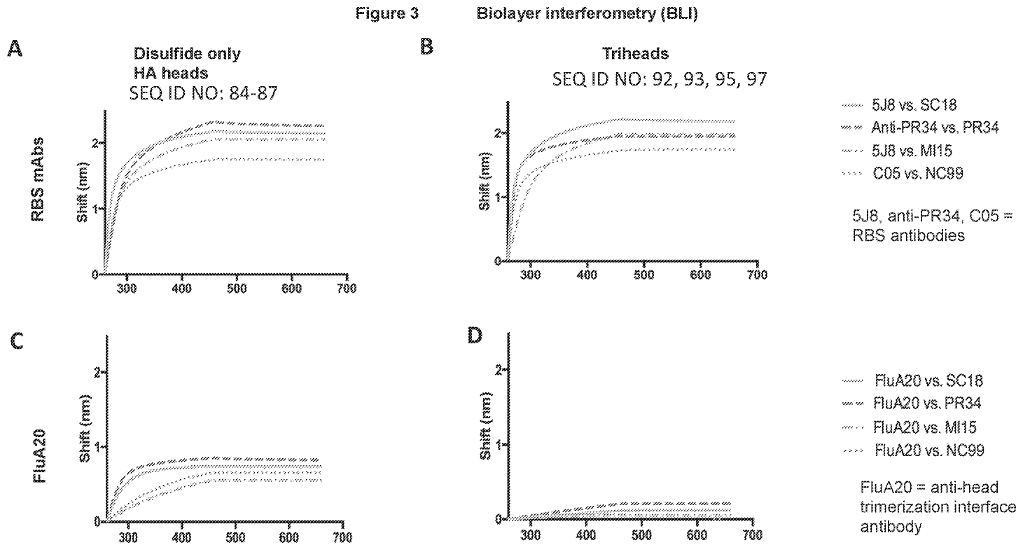

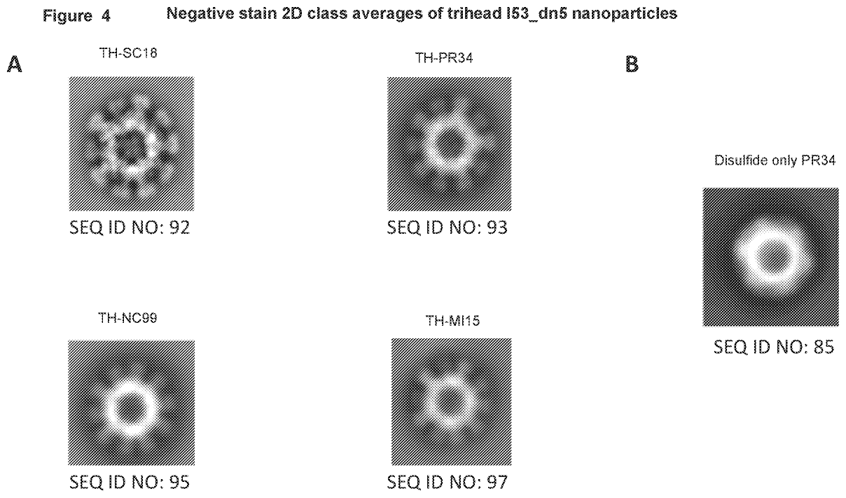

This TriHead design scheme was next adapted into several H1 strains, including A/South Carolina/1/1918 (SEQ ID NO: 92, 98), A/Puerto Rico/9/1934 (SEQ ID NO: 93, 99), and A/Michigan/1/2015 (SEQ ID NO: 97, 101) (FIG. 2A). For creating a stable, closed head-head interface, the engineered disulfide bond in the original NC99 TriHead of SEQ ID NO: 95 was used in the other three strains, as well as some similar mutations at the interface, with some differences as shown in FIG. 2A. Glycans were also added into the side of each of these strains of TriHeads as a means to further focus responses onto the RBS (SEQ ID NOs: 98-101). The stability of all four of these hyperglycosylated strains was demonstrated using thermal melt assays, where all TriHeads (SEQ ID NOs: 98-101) had higher melting temperatures as compared to their counterparts that contained the disulfide bond but lacked the head-head interface mutations (SEQ ID NOs: 88-91) (FIG. 2B). Biolayer interferometry (BLI) was used to show that all TriHeads (SEQ ID NOs: 92, 93, 95, 97) had intact RBS epitopes as they all bound to known RBS-directed monoclonal antibodies (FIG. 3A), but none of them bound to the anti-trimerization interface antibody FluA20 (FIG. 3C). This is in comparison to all corresponding disulfide-only HA head constructs (SEQ ID NOs: 84-87) that lack head-head interface mutations which all bound both RBS mAbs (FIG. 3A) and FluA20 (FIG. 3C). This shows that the interface mutations help rigidly close the HA heads together and thus prevent antibody binding to the trimerization interface. Rigid attachment to a nanoparticle scaffold was also confirmed for all strains of TriHeads (SEQ ID NOs: 92, 93, 95, 97) displayed on I53_dn5 nanoparticles using negative stain 2D class averages, as the HA head density is clearly visible on the surface of the nanoparticle for each TriHead (FIG. 4A) but is not at all for the disulfide-only A/Puerto Rico/9/1934 construct of SEQ ID NO: 85 (FIG. 4B).

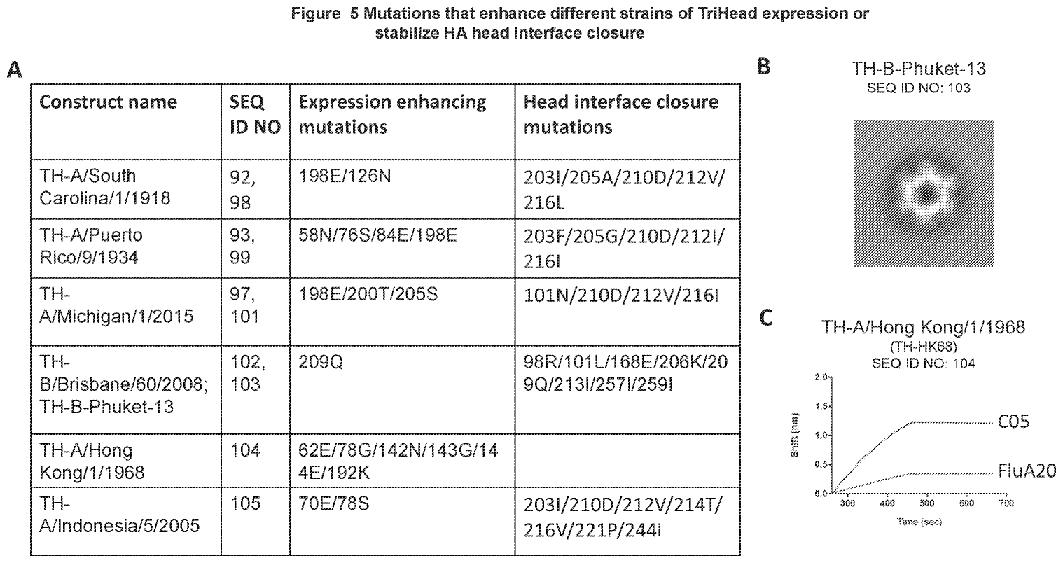

In addition to the H1 strains, TriHead designs have been made for the following strains: H3 A/Hong Kong/1/1968 (SEQ ID NO: 104), H5 A/Indonesia/5/2005 (SEQ ID NO: 105), and type B B/Brisbane/60/2008 (SEQ ID NO: 102) and B-Phuket-13 (SEQ ID NO: 103). Specific mutations that enhanced expression or stabilized head interface closure for these strains and three H1 strains are listed in Table 4. Negative stain EM 2D class average of the B-Phuket-13 (SEQ ID NO: 103) TriHead displayed on the I53_dn5 nanoparticle shows it can be made and the heads are partially stabilized (FIG. 5B). BLI of anti-RBS mAb C05 against H3 Trihead A/Hong Kong/1/1968 (SEQ ID NO: 104) shows it is antigenically intact, with little binding to FluA20, indicative that it may be somewhat stabilized (FIG. 5C).

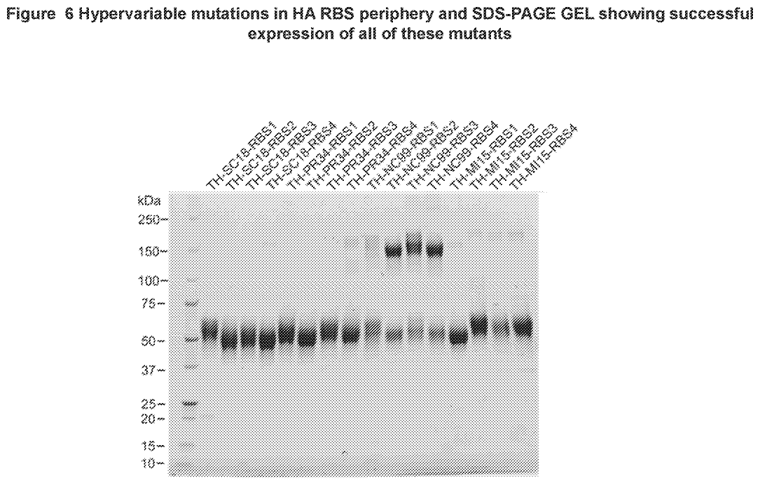

Another layer of immune refocusing consisted of a combinatorial library of amino acid mutations within the HA RBS periphery (FIG. 3) that were made on the H1 hyperglycosylated Trihead constructs (SEQ ID NOs: 98-101). The table of mutations that were made within each the four H1 strains of hyperglycosylated TriHeads is shown in Tables 2 and 4, while FIG. 6 is an SDS-PAGE gel showing that all of these TriHeads could be expressed.

TABLE 4

| Trihead | |
|---|---|
| RBS variant | Mutations |
| TH-SC18-RBS1 | A144E/S145N/K157S/S159D/T189A/Q192T/S193D |
| TH-SC18-RBS2 | A144M/S145N/S159N/T189E/Q192K/K222E/A227I |
| TH-SC18-RBS3 | A144E/S145D/S159K/T189A/Q192E/S193D/K222Q/ |
| | D225N/A227I |
| TH-SC18-RBS4 | S145A/T189E/Q192K/K222E/D225N/A227K |
| TH-PR34-RBS1 | K144Q/S145D/K189S/Q192K/D225G/A227H |
| TH-PR34-RBS2 | E158Q/K189E/Q192T/N193A/K222E/D225G/A227H |
| TH-PR34-RBS3 | E158K/K189N/Q192I/N193S/A227S |
| TH-PR34-RBS4 | Q192H/N193D |
| TH-NC99-RBS1 | G143S/K144V/G189E/R192H/K222A/E227K |
| TH-NC99-RBS2 | K144E/N158G/G159D/G189Q/R192L/A193N/K222V/ |
| | E227Q |
| TH-NC99-RBS3 | K144Q/S145L/N158S/R192K/K222V/D225N/E227Q |
| TH-NC99-RBS4 | K144Q/S145L/G189Q/R192L/A193N/D225N/E227H |
| TH-MI15-RBS1 | A142G/G143N/A144V/A189T/Q192M/S193N |
| TH-MI15-RBS2 | A142G/G143N/A144V/N159D/A189K/Q192A/S193T/ |
| | K222T/Q225G/G227Q |
| TH-MI15-RBS3 | A144E/K145N/N159S/A189K/Q192M/S193N/K222T/ |
| | Q225G/G227Q |
| TH-MI15-RBS4 | A189K/Q192A/S193T |

Structural effects of alterations to the extendable heptad motif were next studied. Using NC99 as the strain, two additional sequences were designed to provide different rigid spacings of TriHeads (FIG. 7A). One construct (SEQ ID NO: 94) (“Closed/Rigid-1”) was designed that removed a heptad repeat from the original Trillead design (SEQ ID NO: 95) (“Closed/Rigid-2”), while a third construct was designed that added four additional heptad repeats to greatly lengthen the extendable region (SEQ ID NO: 96) (“Closed/Rigid-3”). Further, a fourth construct was designed that introduced a Gly-Ser linker directly in between the C-terminal end of the heptad motif and I53_dn5B (SEQ ID NO: 107) (“Closed/Flexible”), which was intended to maintain trimeric closure of RBDs while introducing flexibility between the antigens and the nanoparticle surface. After assembling all designs into nanoparticles, negative stain EM averages were collected for all three new constructs, in addition to a construct based on the original design of SEQ ID NO: 83 but lacking the designed disulfide and inter-RBD interface (“Open/Flexible”). Low-resolution 3D reconstructions of both the Closed/Rigid-1 and Closed/Rigid-3 designs demonstrated significant rigidity between the TriHead antigens and nanoparticle surface, with clearly different TriHead-TriHead spacing (FIG. 7A). In contrast, both the Closed/Flexible and Open/Flexible constructs did not show clear density for the antigens, validating the intended flexibility between antigens and the nanoparticle surface of these constructs.

An immunogenicity study was initiated in mice to understand the effects of both RBD trimeric closure and antigen spacing. BALB/c mice were immunized three times with equimolar doses of nanoparticle immunogens from all five groups (SEQ ID NOs: 83, 94-96, 107), with serum collected two weeks after the third immunization (FIG. 7B). Enzyme-linked immunosorbent assay (ELISA) binding titers against vaccine-matched NC99 HA reached the upper limit of detection, indicating strong immunogenicity of all constructs (FIG. 7C). However, differences in the quality of immunogenicity against NC99 viruses was resolved by testing microneutralization (FIG. 7D). Between all three immunogens with rigidly spaced antigens, stronger neutralizing titers were observed for groups with shorter heptad motif regions. Neutralizing titers elicited by the Closed/Rigid-1 immunogen also far exceeded those elicited by both the Closed/Flexible and Open/Flexible immunogens. Neutralizing antibodies against the RBD most commonly work by blocking receptor binding (Krammer et al. 2020), suggesting that the combination of closure, rigidity and shortened heptad motif region may assist direction of humoral responses towards the apical region of the RBD near the RBS. Breadth of binding titers was also measured against two mismatched H1N1 viruses (FIG. 7C), in which closer spacings similarly demonstrated improvements to vaccine-mismatched antibody responses. In contrast to strain-matched neutralizing titers, maximal breadth of binding was observed by the Open/Flexible group, suggesting that responses against the interface region could assist breadth in ways consistent with broadly cross-reactive interface-directed antibodies. In all, the multiple levels of control provided by the customizability of this platform allows for tuning of responses against the HA head to preferentially include or focus responses to particular epitopes.

### DISCUSSION

The ability to focus antibody responses on arbitrary epitopes has long been desired. Most strategies have aimed to focus antibodies on desirable epitopes by minimizing responses to undesirable epitopes, either by removing or obscuring them through domain deletions or hyperglycosylation. This approach inherently involves removing parts of antigens that could participate in polyclonal responses. The results presented here demonstrate an advance in the structural precision of designed nanoparticle immunogens, and improved vaccines for influenza and other pathogens. To our knowledge, this research describes the most rigid attachment of a glycoprotein antigen to a protein nanoparticle to date. Beyond this, the approach used is particularly versatile in the use of helical bundles with modular units of heptad repeats to mediate between two rigidly attached bodies, which allows for different rigid spacings of antigens on nanoparticle surfaces. The rigidity of and resultant precision of the antigen-antigen spacings achieved, as validated by electron microscopy, demonstrates how geometric properties of immunogens can correlate with their immunogenicity. Given the clear differences in neutralization between distinct rigidly-designed immunogens, it is likely that these geometric differences are responsible for shifting responses to particular epitopes of the head, such as the apical region that features the RBS. This improved precision in the particulate display of antigens is a complementary strategy to epitope-focused immunogen design that does not require direct alterations to intrinsic antigenicity.

For influenza vaccines, the data provides exemplary novel head-domain immunogens, wherein the head-domain epitopes can be substituted with other antigens, such as other viral antigens. Head-directed responses are highly valuable, with HA1 currently the most widely used correlate of protection and commercial vaccines largely relying on antibody responses with measurable HA1 activity. The ability to directly improve the potency and/or breadth of head-directed responses permits improved influenza vaccines. Here, we present modular strategies for altering the immunogenicity of the head domain of multiple strains of HA without modifications to key exterior antigenic regions, which stands in contrast to other published strategies for head-directed vaccines, and which can be extrapolated to diverse vaccine targets.

### Materials and Methods

### Protein Expression and Purification

All designed HA head constructs were expressed by transient transfection in Expi293F cells (ThermoFisher Scientific) at a density of 2.5×10⁶ cells/ml using the ExpiFectamine™ 293 Transfection Kit (ThermoFisher Scientific). The supernatants were harvested 5 days post-transfection and centrifuged at 4000 rpm to remove cell debris. Proteins were purified from clarified supernatants by immobilized metal affinity chromatography (IMAC) using either Ni²⁺- or Co²⁺-containing resin. For Ni²⁺-based IMAC, clarified supernatants were incubated for 2 h at room temperature with Ni²⁺ Sepharose™ High Performance histidine-tagged protein purification resin (Cytiva) and bound protein eluted using 50 mM Tris pH 8.0, 0.5 M NaCl, 300 mM imidazole. For Co²⁺-based resin, clarified supernatants were flowed over Talon™ resin (Takara), and bound protein eluted in 20 mM Tris pH 8.0, 300 mM NaCl, 300 mM imidazole. Eluted proteins were further purified by SEC into phosphate-buffered saline (PBS) or 25 mM Tris pH 8.0, 150 mM NaCl, 5% glycerol using a Superdex™ 200 Increase 10/300 column (Cytiva).

### Negative Stain EM Sample Preparation and Analysis

NS-EM and particle image averaging was used to assess whether the head domains of recombinant NA proteins adopted the open or closed tetrameric structure. Proteins were diluted to between 0.1-0.2 mg/mL using either 10 mM HEPES pH 7.0, 150 mM NaCl or 10 mM Tris pH 7.5, 150 mM NaCl. Samples were adsorbed to glow-discharged carbon-coated copper grids. The grids were either washed with a drop of the same buffer three times and stained with 0.75% uranyl formate, or blotted and stained directly with 0.75% uranyl formate. Images were recorded with sampling ranging between 1.9 Å/pixel and 2.2 Å/pixel, depending on the microscope. Data were collected on either an FEI Tecnai™ T20 electron microscope equipped with an FEI Eagle CCD camera and operated at 200 kV using SerialEM (Takaba, Maki-Yonekura, and Yonekura 2020), or on an FEI Tecnai™ 12 Spirit 120 kV electron microscope equipped with a Gatan Ultrascan™ 4000 CCD camera. Particles were selected from the micrographs automatically using either in-house software (Yaroslav Tsybovsky, unpublished) or were picked in a reference-free manner in cisTEM (Grant, Rohou, and Grigorieff 2018). For the latter datasets, particles were extracted after correcting for the effect of the CTF for each micrograph with cisTEM (Grant, Rohou, and Grigorieff 2018). Depending on user, dataset, and microscope, particles were either extracted into 120×120-pixel boxes with a final pixel size of 2.2 Å/pixel or extracted with a box size of 176×176 pixels and binned to a final box size of 44×44 pixels (to a pixel size of 6.4 Å/pixel). Reference-free 2D classification was performed using either Relion 1.4 or Relion 3.1 (Zivanov et al. 2018; Scheres 2015).

### Protein-Protein Interface Design Using Rosetta™

All calculations in Rosetta™ were made using versions v2017.18-dev59451, v2019.21-dev60746, or v2019.45-dev61026. For design of interfaces between HA heads, residue positions were manually selected in the head-head interface of a model of a closely-related strain to NC99 (PDB ID 5UG0). The three-fold symmetry axis of the symmetric HA trimer was aligned with [0,0,1] and a single protomer was saved in .pdb format. A design protocol was written using RosettaScripts™ (Fleishman et al. 2011) that takes the aligned protomer and a custom resfile as inputs, with the resfile dictating the side chain identities and conformations sampled during design. Briefly, the protocol applies two rounds of design based on the input resfile, with side chain and backbone energy minimization applied after each design step. Both design and minimization steps were allowed to repack or minimize residues within 5 Å of all mutable or packable residues listed in the resfile. Multiple resfiles were set up to diversify allowed residue identities at each position to lead trajectories towards different solutions. Design models and scores were manually inspected to identify interactions across the interface that appeared structurally feasible. Favorable interactions were iteratively retested in resfiles and manually refined to finalize a diverse set of designs.

### Thermal Melts

Thermal melt assays using SYPRO dye were performed on an UNcle (Unchained Labs). All proteins were mixed with SYPRO Orange at 1:2000 and then the change in SYPRO fluorescence was monitored as the dye binds to exposed hydrophobics in proteins with increasing temperature.

### Biolayer Interferometry

BLI binding assays were performed on an Octet Red instrument at 25° C. with shaking at 1,000 RPM in the presence of 25 mM Tris pH 8.0, 150 mM NaCl and 5% glycerol. Anti-hIgG Capture (AHC) tips were loaded with human D2 H1-1/H3-1 or COS at 0.02 mg/mL for 300 s prior to a baseline for 60 s, association with trimeric components at 500 nM for 600 s, and dissociation for 300 s.

### Cryo-Electron Microscopy Sample Preparation, Data Collection, and Image Processing

Nanoparticle samples were diluted to 1-1.5 μM in buffer (10 mM Tris, pH 7.5, 150 mM NaCl) and 3 μL sample loaded onto a freshly glow-discharged 1.2/1.2 UltrAuFoil grid (300 mesh) prior to plunge freezing using a vitrobot Mark IV (ThermoFisher Scientific) with a blot force of −1 and 3.5-4.5 s blot time at 100% humidity and 4° C. Data were acquired on an FEI Glacios™ transmission electron microscope operated at 200 kV and equipped with a Gatan K2 Summit direct detector. Automated data collection was carried out using Leginon™ (Suloway et al. 2005) at a nominal magnification of 36,000× with a pixel size of 1.16 Å. The dose rate was adjusted to 8 counts/pixel/s, and each movie was acquired in counting mode fractionated in 50 frames of 200 ms. Reference-free 2D and 3D classification were performed using cryoSPARC™ (Punjani et al. 2017).

### REFERENCES

Allen, James D., Satyajit Ray, and Ted M. Ross. 2018. “Split Inactivated COBRA Vaccine Elicits Protective Antibodies against H1N1 and H3N2 Influenza Viruses.” PloS One 13 (9): e0204284.
Bachmann, Martin F., and Gary T. Jennings. 2010. “Vaccine Delivery: A Matter of Size, Geometry, Kinetics and Molecular Patterns.” Nature Reviews. Immunology 10 (11): 787-96.
Bachmann, M. F., and R. M. Zinkernagel. 1996. “The Influence of Virus Structure on Antibody Responses and Virus Serotype Formation.” Immunology Today 17 (12): 553-58. 1997. “Neutralizing Antiviral B Cell Responses.” Annual Review of Immunology 15:235-70.
Bajic, Goran, Max J. Maron, Yu Adachi, Taishi Onodera, Kevin R. McCarthy, Charles E. McGee, Gregory D. Sempowski, et al. 2019. “Influenza Antigen Engineering Focuses Immune Responses to a Subdominant but Broadly Protective Viral Epitope.” Cell Host & Microbe 25 (6): 827-35.e6.
Bangaru, Sandhya, Shanshan Lang, Michael Schotsaert, Hillary A. Vanderven, Xueyong Zhu, Nurgun Kose, Robin Bombardi, et al. 2019. “A Site of Vulnerability on the Influenza Virus Hemagglutinin Head Domain Trimer Interface.” Cell 177 (5): 1136-52.e18.
Bangaru, Sandhya, Heng Zhang, Juliia M. Gilchuk, Thomas G. Voss, Ryan P. Irving, Pavlo Gilchuk, Pranathi Matta, et al. 2018. “A Multifunctional Human Monoclonal Neutralizing Antibody That Targets a Unique Conserved Epitope on Influenza HA.” Nature Communications 9 (1): 2669.
Belongia, Edward A., Melissa D. Simpson, Jennifer P. King, Maria E. Sundaram, Nicholas S. Kelley, Michael T. Osterholm, and Huong Q. McLean. 2016. “Variable Influenza Vaccine Effectiveness by Subtype: A Systematic Review and Meta-Analysis of Test-Negative Design Studies.” The Lancet Infectious Diseases 16 (8): 942-51.
Benton, Donald J., Steven J. Gamblin, Peter B. Rosenthal, and John J. Skehel. 2020. “Structural Transitions in Influenza Haemagglutinin at Membrane Fusion pH.” Nature 583 (7814): 150-53.
Broecker, Felix, Sean T. H. Liu, Nungruthai Suntronwong, Weina Sun, Mark J. Bailey, Raffael Nachbagauer, Florian Krammer, and Peter Palese. 2019. “A Mosaic Hemagglutinin-Based Influenza Virus Vaccine Candidate Protects Mice from Challenge with Divergent H3N2 Strains.” NPJ Vaccines 4 (July): 31.
Brouwer, Philip J. M., Aleksandar Antanasijevic, Zachary Berndsen, Anila Yasmeen, Brooke Fiala, Tom P. L. Bijl, Ilja Bontjer, et al. 2019. “Enhancing and Shaping the Immunogenicity of Native-like HIV-1 Envelope Trimers with a Two-Component Protein Nanoparticle.” Nature Communications 10 (1): 4272.
Das, Dibyendu Kumar, Ramesh Govindan, Ivana Nikić-Spiegel, Florian Krammer, Edward A. Lemke, and James B. Munro. 2018. “Direct Visualization of the Conformational Dynamics of Single Influenza Hemagglutinin Trimers.” Cell 174 (4): 926-37.e12.
Dintzis, H. M., R. Z. Dintzis, and B. Vogelstein. 1976. “Molecular Determinants of Immunogenicity: The Immunon Model of Immune Response.” Proceedings of the National Academy of Sciences of the United States of America 73 (10): 3671-75.
Ekiert, Damian C., Arun K. Kashyap, John Steel, Adam Rubrum, Gira Bhabha, Reza Khayat, Jeong Hyun Lee, et al. 2012. “Cross-Neutralization of Influenza A Viruses Mediated by a Single Antibody Loop.” Nature 489 (7417): 526-32.
Fleishman, Sarel J., Andrew Leaver-Fay, Jacob E. Corn, Eva-Maria Strauch, Sagar D. Khare, Nobuyasu Koga, Justin Ashworth, et al. 2011. “RosettaScripts: A Scripting Language Interface to the Rosetta Macromolecular Modeling Suite.” PloS One 6 (6): e20161.
Giles, Brendan M., and Ted M. Ross. 2011. “A Computationally Optimized Broadly Reactive Antigen (COBRA) Based HSNI VLP Vaccine Elicits Broadly Reactive Antibodies in Mice and Ferrets.” Vaccine 29 (16): 3043-54.
Grant, Timothy, Alexis Rohou, and Nikolaus Grigorieff. 2018. “cisTEM, User-Friendly Software for Single-Particle Image Processing.” eLife 7 (March): e35383.
Harbury, P. B., P. S. Kim, and T. Alber. 1994. “Crystal Structure of an Isoleucine-Zipper Trimer.” Nature 371 (6492): 80-83.
Harris, Audray K., Joel R. Meyerson, Yumiko Matsuoka, Oleg Kuybeda, Amy Moran, Donald Bliss, Suman R. Das, et al. 2013. “Structure and Accessibility of HA Trimers on Intact 2009 H1N1 Pandemic Influenza Virus to Stem Region-Specific Neutralizing Antibodies.” Proceedings of the National Academy of Sciences of the United States of America 110 (12): 4592-97.
Harrison, Stephen C. 2015. “Viral Membrane Fusion.” Virology 479-480 (May): 498-507.
Hong, Minsun, Peter S. Lee, Ryan M. B. Hoffman, Xueyong Zhu, Jens C. Krause, Nick S. Laursen, Sung-Il Yoon, et al. 2013. “Antibody Recognition of the Pandemic H1N1 Influenza Virus Hemagglutinin Receptor Binding Site.” Journal of Virology 87 (22): 12471-80.
Impagliazzo, Antonietta, Fin Milder, Harmjan Kuipers, Michelle V. Wagner, Xueyong Zhu, Ryan M. B. Hoffman, Ruud van Meersbergen, et al. 2015. “A Stable Trimeric Influenza Hemagglutinin Stem as a Broadly Protective Immunogen.” Science 349 (6254): 1301-6.
Irvine, Darrell J., and Benjamin J. Read. 2020. “Shaping Humoral Immunity to Vaccines through Antigen-Displaying Nanoparticles.” Current Opinion in Immunology 65 (August): 1-6.
Kanekiyo, Masaru, M. Gordon Joyce, Rebecca A. Gillespie, John R. Gallagher, Sarah F. Andrews, Hadi M. Yassine, Adam K. Wheatley, et al. 2019. “Mosaic Nanoparticle Display of Diverse Influenza Virus Hemagglutinins Elicits Broad B Cell Responses.” Nature Immunology 20 (3): 362-72.
Klein, Joshua S., and Pamela J. Bjorkman. 2010. “Few and Far between: How HIV May Be Evading Antibody Avidity.” PLOS Pathogens 6 (5): €1000908.
Knight, Matthew, Siriruk Changrob, Lei Li, and Patrick C. Wilson. 2020. “Imprinting, Immunodominance, and Other Impediments to Generating Broad Influenza Immunity.” Immunological Reviews 296 (1): 191-204.
Krammer, Florian, and Peter Palese. 2015. “Advances in the Development of Influenza Virus Vaccines.” Nature Reviews. Drug Discovery 14 (3): 167-82.
Krammer, Florian, Jerry P. Weir, Othmar Engelhardt, Jacqueline M. Katz, and Rebecca Jane Cox. 2020. “Meeting Report and Review: Immunological Assays and Correlates of Protection for next-Generation Influenza Vaccines.” Influenza and Other Respiratory Viruses 14 (2): 237-43.
Lee, Jiwon, Daniel R. Boutz, Veronika Chromikova, M. Gordon Joyce, Christopher Vollmers, Kwanyee Leung, Andrew P. Horton, et al. 2016. “Molecular-Level Analysis of the Serum Antibody Repertoire in Young Adults before and after Seasonal Influenza Vaccination.” Nature Medicine 22 (12): 1456-64.
Lee, Peter S., Nobuko Ohshima, Robyn L. Stanfield, Wenli Yu, Yoshitaka Iba, Yoshinobu Okuno, Yoshikazu Kurosawa, and Ian A. Wilson. 2014. “Receptor Mimicry by Antibody F045-092 Facilitates Universal Binding to the H3 Subtype of Influenza Virus.” Nature Communications 5 (April): 3614.
Lee, Peter S., Reiko Yoshida, Damian C. Ekiert, Naoki Sakai, Yasuhiko Suzuki, Ayato Takada, and Ian A. Wilson. 2012. “Heterosubtypic Antibody Recognition of the Influenza Virus Hemagglutinin Receptor Binding Site Enhanced by Avidity.” Proceedings of the National Academy of Sciences of the United States of America 109 (42): 17040-45.
Punjani, Ali, John L. Rubinstein, David J. Fleet, and Marcus A. Brubaker. 2017. “cryoSPARC: Algorithms for Rapid Unsupervised Cryo-EM Structure Determination.” Nature Methods 14 (3): 290-96.
Raymond, Donald D., Goran Bajic, Jack Ferdman, Pirada Suphaphiphat, Ethan C. Settembre, M. Anthony Moody, Aaron G. Schmidt, and Stephen C. Harrison. 2018. “Conserved Epitope on Influenza-Virus Hemagglutinin Head Defined by a Vaccine-Induced Antibody.” Proceedings of the National Academy of Sciences of the United States of America 115 (1): 168-73.
Rimmelzwaan, Guus F., and Janet E. McElhaney. 2008. “Correlates of Protection: Novel Generations of Influenza Vaccines.” Vaccine 26 Suppl 4 (September): D41-44.
Sautto, Giuseppe A., Greg A. Kirchenbaum, and Ted M. Ross. 2018. “Towards a Universal Influenza Vaccine: Different Approaches for One Goal.” Virology Journal 15 (1): 17.
Scheres, Sjors H. W. 2015. “Semi-Automated Selection of Cryo-EM Particles in RELION-1.3.” Journal of Structural Biology 189 (2): 114-22.
Schiller, John, and Bryce Chackerian, 2014. “Why HIV Virions Have Low Numbers of Envelope Spikes: Implications for Vaccine Development.” PLOS Pathogens 10 (8): e1004254.
Schmidt, Aaron G., Huafeng Xu, Amir R. Khan, Timothy O'Donnell, Surender Khurana, Lisa R. King, Jody Manischewitz, et al. 2013. “Preconfiguration of the Antigen-Binding Site during Affinity Maturation of a Broadly Neutralizing Influenza Virus Antibody.” Proceedings of the National Academy of Sciences of the United States of America 110 (1): 264-69.
Shaw, Alan, Ian T. Hoffecker, Ioanna Smyrlaki, Joao Rosa, Algirdas Grevys, Diane Bratlie, Inger Sandlie, Terje Einar Michaelsen, Jan Terje Andersen, and Björn Högberg. 2019. “Binding to Nanopatterned Antigens Is Dominated by the Spatial Tolerance of Antibodies.” Nature Nanotechnology 14 (2): 184-90.
Suloway, Christian, James Pulokas, Denis Fellmann, Anchi Cheng, Francisco Guerra, Joel Quispe, Scott Stagg, Clinton S. Potter, and Bridget Carragher. 2005. “Automated Molecular Microscopy: The New Leginon System.” Journal of Structural Biology 151 (1): 41-60.
Sun, Weina, Ericka Kirkpatrick, Megan Ermler, Raffael Nachbagauer, Felix Broecker, Florian Krammer, and Peter Palese. 2019. “Development of Influenza B Universal Vaccine Candidates Using the ‘Mosaic’ Hemagglutinin Approach.” Journal of Virology 93 (12). doi.org/10.1128/JVI.00333-19.
Takaba, Kiyofumi, Saori Maki-Yonekura, and Koji Yonekura. 2020. “Collecting Large Datasets of Rotational Electron Diffraction with ParallEM and SerialEM.” Journal of Structural Biology 211 (2): 107549.
Turner, Hannah L., Jesper Pallesen, Shanshan Lang, Sandhya Bangaru, Sarah Urata, Sheng Li, Christopher A. Cottrell, et al. 2019. “Potent Anti-Influenza H7 Human Monoclonal Antibody Induces Separation of Hemagglutinin Receptor-Binding Head Domains.” PLOS Biology 17 (2): e3000139.
Ueda, George, Aleksandar Antanasijevic, Jorge A. Fallas, William Sheffler, Jeffrey Copps, Daniel Ellis, Geoffrey B. Hutchinson, et al. 2020. “Tailored Design of Protein Nanoparticle Scaffolds for Multivalent Presentation of Viral Glycoprotein Antigens.” eLife 9 (August)./doi.org/10.7554/eLife.57659.
Veneziano, Rémi, Tyson J. Moyer, Matthew B. Stone, Eike-Christian Wamboff, Benjamin J. Read, Sayak Mukherjee, Tyson R. Shepherd, et al. 2020. “Role of Nanoscale Antigen Organization on B-Cell Activation Probed Using DNA Origami.” Nature Nanotechnology 15 (8): 716-23.
Watanabe, Akiko, Kevin R. McCarthy, Masayuki Kuraoka, Aaron G. Schmidt, Yu Adachi, Taishi Onodera, Keisuke Tonouchi, et al. 2019. “Antibodies to a Conserved Influenza Head Interface Epitope Protect by an IgG Subtype-Dependent Mechanism.” Cell 177 (5): 1124-35.c16.
Whittle, James R. R., Ruijun Zhang, Surender Khurana, Lisa R. King, Jody Manischewitz, Hana Golding, Philip R. Dormitzer, et al. 2011. “Broadly Neutralizing Human Antibody That Recognizes the Receptor-Binding Pocket of Influenza Virus Hemagglutinin.” Proceedings of the National Academy of Sciences of the United States of America 108 (34): 14216-21.
Wu, Nicholas C., and Ian A. Wilson. 2020. “Influenza Hemagglutinin Structures and Antibody Recognition.” Cold Spring Harbor Perspectives in Medicine 10 (8). doi.org/10.1101/cshperspect.a038778.
Xu, Rui, Jens C. Krause, Ryan McBride, James C. Paulson, James E. Crowe Jr, and Ian A. Wilson. 2013. “A Recurring Motif for Antibody Recognition of the Receptor-Binding Site of Influenza Hemagglutinin.” Nature Structural & Molecular Biology 20 (3): 363-70.
Zhu, Xueyong, Yong-Hui Guo, Tao Jiang, Ya-Di Wang, Kwok-Hung Chan, Xiao-Feng Li, Wenli Yu, et al. 2013. “A Unique and Conserved Neutralization Epitope in H5N1 Influenza Viruses Identified by an Antibody against the A/Goose/Guangdong/1/96 Hemagglutinin.” Journal of Virology 87 (23): 12619-35.
Zivanov, Jasenko, Takanori Nakane, Björn O. Forsberg, Dari Kimanius, Wim Jh Hagen, Erik Lindahl, and Sjors Hw Scheres. 2018. “New Tools for Automated High-Resolution Cryo-EM Structure Determination in RELION-3.” eLife 7 (November): e42166.

1. A polypeptide, comprising:
(a) a first domain comprising a heptad motif comprising an amino acid sequence according to the genus (I-X1-X2-I-X3-X4-X5)ₙ, wherein X1, X2, X3, X4, and X5 may independently be any amino acid other than proline, and wherein n can be 1-30; and
(b) a second domain selected from the group consisting of:
(i) a polypeptide antigen, and
(ii) a polypeptide component of a nanoparticle.

2. The polypeptide of claim 1, wherein X1 is selected from the group consisting of A, D, E, H, K, N, Q, R, S, Y, and T; or wherein X1 is selected from the group consisting of E, Y, A, and R.

3. The polypeptide of claim 1, wherein X2 is selected from the group consisting of A, D, E, H, K, N, Q, R, S, and T; or wherein X2 is selected from the group consisting of E, H, R, and N.

4. The polypeptide of claim 1, wherein X3 is selected from the group consisting of A, D, E, H, K, L, N, Q, R, S, and T; or wherein X3 is selected from the group consisting of L, E, K, and N.

5. The polypeptide of claim 1, wherein X4 is selected from the group consisting of A, D, E, H, K, N, Q, R, S, and T; or wherein X4 is selected from the group consisting of S, D, N, and K.

6. The polypeptide of claim 1, wherein X5 is selected from the group consisting of A, D, E, H, K, L, N, Q, R, S, and T; or wherein X5 is selected from the group consisting of K, E, and L.

7. The polypeptide of claim 1, wherein the first domain comprises the amino acid sequence selected from the group consisting of SEQ ID NO:6-27, or selected from the group consisting of SEQ ID NO:6 and 8-27, or selected from the group consisting of SEQ ID NO:6, 8-25, and 27, or selected from the group consisting of SEQ ID NO:6 and 13.

8. The polypeptide of claim 1, wherein the second domain comprises a polypeptide antigen.

9. (canceled)

10. The polypeptide of claim 8, wherein the polypeptide antigen comprises an influenza hemagglutinin (HA) protein, or immunogenic portion thereof including but not limited to a HA head domain, a HA receptor binding domain (RBD), and/or a HA apical receptor binding site (RBS), or immunogenic portion thereof.

11. The polypeptide of claim 10, wherein the HA protein or immunogenic portion thereof comprises an amino acid sequence at least 75%, 80%, 85%, 90%, 91%, 92%, 93%, 94%, 95%, 96%, 97%, 98%, or 99% identical to the amino acid sequence of SEQ ID NO: 1 residues 51-264 (the head domain, bolded in SEQ ID NO:1), wherein the HA protein or immunogenic portion thereof includes 1, 2, 3, 4, or all 5 of the following amino acid residues relative to SEQ ID NO:1 when aligned by protocol 1 or protocol 2: 107C, 203L, 210D, 212V or I (or 212V), and/or 216I, wherein residues in parentheses are not present in mature HA protein.

12.-23. (canceled)

24. The polypeptide of claim 1, wherein the second domain comprises a polypeptide component of a nanoparticle.

25. (canceled)

26. The polypeptide of claim 24, wherein the polypeptide component of a nanoparticle comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:114.

27.-28. (canceled)

29. The polypeptide of claim 24, further comprising a third domain comprising a polypeptide antigen.

30.-34. (canceled)

35. The polypeptide of claim 1 wherein the polypeptide comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO:83-113.

36. A mutated HA polypeptide comprising an amino acid sequence at least 75%, 80%, 85%, 90%, 91%, 92%, 93%, 94%, 95%, 96%, 97%, 98%, or 99% identical to the amino acid sequence of SEQ ID NO:1, wherein the HA protein or immunogenic portion thereof includes 1, 2, 3, 4, or all 5 of the following amino acid residues relative to SEQ ID NO:1 residues 51-264 (the head domain, bolded in SEQ ID NO:1) when aligned by protocol 1 or protocol 2:
107C, 203L, 210D, 212V or I (or 212V), and/or 216I.

37.-48. (canceled)

49. A nucleic acid encoding the polypeptide of claim 1.

50.-56. (canceled)

57. An expression vector, comprising the nucleic acid of claim 49, operatively linked to a suitable control sequence.

58. A host cell comprising the expression vector of claim 57.

59. (canceled)

60. A nanoparticle comprising a plurality of the polypeptides of claim 1.

61.-84. (canceled)

85. A method to vaccinate a subject against an infectious agent, including but not limited to the influenza virus, the method comprising administering to the subject the nanoparticle of claim 60.

86.-92. (canceled)
